(19) United States
(12) Patent Application Publication
(10) Pub. No.: US 2008/0193397 A1
(43) Pub. Date: Aug. 14, 2008

(54) Use of Peroxide Decomposers Method For the Treatment of a Material Other Than the Human Body

(75) Inventors: Manfred Kroll, Ludwigshafen (DE); Gerhard Wagenblast, Wachenheim (DE); Arne Ptock, Ludwigshafen (DE); Sylke Haremza, Neckargemund (DE); Axel Jentzsch, Ludwigshafen (DE)

Correspondence Address: CONNOLLY BOVE LODGE & HUTZ LLP, 1875 EYE STREET, N.W., SUITE 1100, WASHINGTON, DC, 20036, US

(73) Assignee: BASF Aktiengesellscaft, Ludwigshafen (DE)

(21) Appl. No.: 11/914,186

(22) PCT Filed: May 12, 2006
(86) PCT No.: PCT/EP2006/004487

§ 371 Date: Nov. 12, 2007

(30) Foreign Application Priority Data
May 13, 2005 (DE) 102005022292.7

(51) Int. Cl. A61K 8/46; A61K 31/426; A61K 31/505; A61K 31/54; A61K 31/53; A61K 31/4412; A61K 31/4196; A61K 31/44

(52) U.S. Cl. 424/59; 514/369; 514/274; 514/224.2; 514/345; 514/384; 514/350; 514/242

(57) ABSTRACT

The invention relates to the use of compounds with certain sulfur and optionally nitrogen atom-containing structures as peroxide decomposers in cosmetic and pharmaceutical agents for treating the skin. The invention also relates to cosmetic and pharmaceutical agents comprising compounds having such structures.

The present invention relates to the use of compounds with certain sulfur- and if appropriate nitrogen-atom-containing structures as peroxide decomposers in cosmetic and pharmaceutical agents for treating skin. The invention further relates to cosmetic and pharmaceutical agents which comprise compounds having such structures.

Human skin is subject to certain aging processes some of which are attributable to intrinsic processes (chronoaging) and some of which are attributable to exogenous factors (environmental, e.g. photoaging). In addition, temporary and also lasting changes in the appearance of the skin arise, such as acne, greasy or dry skin, keratoses, rosaceae, photosensitive, inflammatory erythematous, allergic or autoimmune reactive reactions, such as dermatoses and photodermatoses and others, whose precise causes and factors which influence them are often understood only incompletely.

The exogenous factors include, in particular, sunlight or artificial sources of radiation with a comparable spectrum, and also compounds which can arise due to radiation, such as undefined reactive photoproducts, which may also be free-radical or ionic. However, these factors also include harmful or reactive compounds such as ozone, free radicals, for example the hydroxyl radical, singlet oxygen and other reactive oxygen or nitrogen compounds, cigarette smoke, natural and synthetic toxins, and others which disturb the natural physiology or morphology of the skin. The effect of these factors can, inter alia, lead to direct damage to the DNA of the skin cells, and of the collagen, elastin or glycosaminoglycan molecules in the extracellular matrix which are responsible for the strength of the skin. Moreover, the result may be an influence on the signal transduction chains at the end of which the activation of harmful factors, e.g. matrix-degrading enzymes, stands. Important representatives of these enzymes are the matrix metalloproteinases (MMPs, e.g. collagenases, gelatinases, stromelysins), the activity of which is additionally regulated by TIMPs (tissue inhibitor of matrix metalloproteinases).

Furthermore, the harmful effects lead to damage to the cells of the skin itself. As a consequence of this, the ability of the skin to regenerate, for example, is reduced.

A further consequence may be inflammatory reactions, and, inter alia, immunoregulatory compounds, such as interleukins, prostaglandins and histamines are released. As a result, immunocompetent cells are attracted, inter alia, and the inflammatory reaction is intensified.

The consequences of aging are thinning of the skin, weaker meshing of epidermis and dermis, reduction in cell number and in supplying blood vessels. The aging processes lead to the formation of fine lines and wrinkles, the skin becomes leathery, yellowish and starts to sag, and pigment disorders arise.

Antioxidants effective as free-radical scavengers are often used in dermatological or cosmetic preparations for protecting against decay. Moreover, they can, however, also be used in order to reduce harmful or undesired oxidative processes which occur in human or animal skin. Such processes are known to play a significant role in skin aging. The skin is permanently exposed to oxidative stress through the formation of peroxides and hydroperoxides, some of which originate from the external environment of the skin, but some of which are also formed endogenously. In order to counteract this stress, the skin has a large number of its own protective mechanisms. These protective mechanisms, however, are insufficient to completely prevent oxidative processes in the skin. On the contrary, it is generally assumed that these very oxidative processes make a significant contribution to skin aging, but also to general or pathological changes in the skin.

In particular, the importance of lipid peroxidation for aging is generally recognized. The toxic effect of lipid hydroperoxides and their decomposition products has been described, inter alia, by W. A. Prior (ACS Sysup. Ser. (1985), 277, 77-96).

K. Chiba et al. describe, in Skin Pharmacol. Appl. Skin Physiol., 2003, 16, 242-251, the formation of skin wrinkles and other skin changes induced by repeated exposure to squalene monohydroperoxide on bald mice.

Ekanayake et al. describe, in The Journal of Investigative Dermatology, Volume 120, 2003, 6, 915-922, the UVA-induced formation of squalene hydroperoxide isomers in the human sebum and in lipids in the surface of the skin.

There is further a great need for compounds which are able to effectively even prevent the formation of peroxides and hydroperoxides when used in topical formulations.

WO 98/51268 describes hair colorants which comprise at least one pyrazoline-4,5-dione and at least one aromatic or heteroaromatic amine. This amine may, for example, be a thiouracil.

DE-A-100 24 886 describes an: agent for coloring keratin-containing fibers which comprises at least one nitrosopyridine and/or -pyrimidine. These compounds can be substituted, inter alia, by mercapto groups.

DE-A-25 23 629 describes a hair colorant which comprises a 5,6-diamino-4-oxopyrimidine derivative which can comprise a double-bonded sulfur atom in the 2-position of the heterocycle.

EP-A-0 422 765 describes a topical composition for promoting hair growth, where the hair growth promoters used are, inter alia, nitrogen- and sulfur-containing heterocycles, such as, for example, 5-carboxy-5-hydroxyvalero-1,4-thiolactone.

EP-A-0 540 854 describes a composition promoting hair growth which comprises, as active ingredient, at least one compound which is selected from purines, pyridylureas, diphenylureas, pyrimidines, imidazoles, benzoylaminoureas and 4-substituted aminopyrrole[2,3-d]pyrinidines. Besides a large number of further substituents, these can, inter alia, also carry thio groups.

EP-A-0 483 426 describes a deodorant composition which comprises a heterocyclic thiol as active ingredient.

CN-A-1075180 describes the bactericidal, deodorizing finishing of fabrics where, inter alia, thiols and thiocarboxylic acids are used.

JP 05124924-A describes a topical medicament for inhibiting melamine production which comprises at least one pyridine- or pyrimidinethiol as active ingredient.

U.S. Pat. No. 6,383,476 describes anhydrous antiperspirant and deodorant compositions which comprise, inter alia, nitrogen heterocycles substituted by sulfur-containing groups, such as thionicotinamide and 2-mercaptothionicotinic acid, as active ingredient.

WO 01/64206 describes a method of inhibiting or reducing pigment production in a mammal in which the active ingredient used is a six-membered ring heterocycle which carries, as one substituent, a structure derived from an amide or thioamide, which may be, inter alia, nitrogen-atom-containing heterocycles.

WO 02/053101 describes a method of treating fibrotic disorders where the active ingredient used is certain nitrogen heterocycles which can have sulfur atoms or sulfur-atom-containing groups in the ring or as substituents and where this active ingredient can be administered topically.

None of the abovementioned documents describes a use of the sulfur-atom-containing compounds disclosed therein as peroxide decomposers in cosmetic and pharmaceutical agents for treating skin.

DE-A-1 02 02 065 describes cosmetic or dermatological compositions which comprise a) at least one antioxidant effective as O or C free-radical scavenger, and b) at least one organic boron-comprising compound as peroxide decomposer.

DE-A-100 36 655.4 describes the use of a combination of a) at least one antioxidant effective as O or C free-radical scavenger and b) at least one organic or inorganic skin-compatible compound which reduces peroxides or hydroperoxides to the corresponding alcohols without formation of reactive free-radical subsequent states, as additive for cosmetic or dermatological preparations for avoiding skin damage by peroxides or hydroperoxides formed as a consequence of endogenous or exogenous factors.

It is an object of the present invention to provide active ingredients for use as peroxide decomposers in cosmetic or pharmaceutical preparations for treating skin which are characterized by good effectiveness against the formation of free radicals that are harmful to the skin.

The present invention therefore provides the use of compounds of the general formulae I.1 to I.3

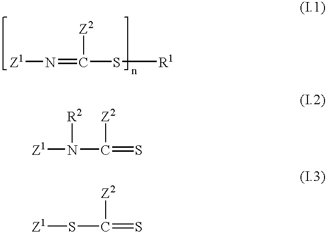

in which
n is an integer from 1 to 3,
at least one of the radicals Z¹ and Z² is an organyl radical and the other is hydrogen or an organyl radical, where Z¹ and Z² may also together be a divalent bridging group having 1 to 5 atoms between the flanking bonds,
R¹ is hydrogen, alkyl, cycloalkyl, heterocycloalkyl, aryl, heteroaryl or an n-valent cation, and
R² is hydrogen, alkyl, cycloalkyl, heterocycloalkyl, aryl, hetaryl, COOR^{a}, COO⁻M⁺, SO₃R^{a}, SO₃⁻M⁺, sulfonamide, NE¹E², (NE¹E²E³)⁺A⁻, OR^{a}, SR^{a}, (CHR^{b}CH₂O)_{y}R^{a}, (CH₂O)_{y}R^{a}, (CH₂CH₂NE¹)_{y}R^{a}, alkylaminocarbonyl, dialkylaminocarbonyl, alkylcarbonylamino or acyl, where
   R^{a} are in each case identical or different radicals selected from hydrogen, alkyl, cycloalkyl, aryl or hetaryl,
   E¹, E², E³ are in each case identical or different radicals selected from hydrogen, alkyl, cycloalkyl, aryl or hetaryl,
   R^{b} is hydrogen, methyl or ethyl,
   M⁺ is a cation equivalent,
   A⁻ is an anion equivalent and
   y is an integer from 1 to 250,

as peroxide decomposer in cosmetic and pharmaceutical agents for treating skin.

For the purposes of the present invention, a “peroxide decomposer” is understood as meaning an antioxidant which, in contrast to customary free-radical scavengers, even avoids or at least reduces the formation of peroxides and/or hydroperoxides.

In general, the mechanism of the formation of peroxide or hydroperoxide conforms to the following scheme

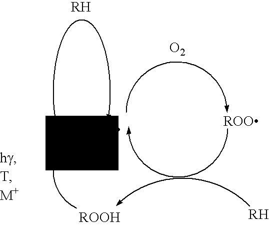

While the customary antioxidants are essentially O or C free-radical scavengers the compounds of the general formulae I.1 to I.3 used according to the invention are capable, by intervening in the mechanism of the scheme depicted above, of efficiently preventing skin damage additionally at another site. Advantageously, these compounds are effective in an early stage of free-radical formation, meaning that multiple free-radical formation is prevented. This can take place, for example, on the basis of an ionic and reductive attack according to the following scheme, although other modes of activity are also conceivable.

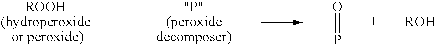

The compounds of the general formulae I.1 to I.3 used according to the invention also include resonance-stabilized compounds, e.g. aromatic compounds in which only individual resonance structures (mesomeric limiting structures) are given by the formulae I.1 to I.3.

Of suitability for the use according to the invention are, advantageously, compounds of the formulae I.1 to I.3 which are capable of tautometry. By way of example, such a tautometry equilibrium is shown below:

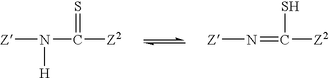

It has been found that the compounds of the formulae I.1 to I.3 used according to the invention are characterized by a significantly greater peroxide and/or hydroperoxide decomposing (reducing) effect than endogenous sulfur-comprising compounds, such as cystine or cysteine. They are generally able, in the form of a solution with a molar concentration of 0.05 mol/l in a polar or nonpolar solvent, to reduce a starting peroxide or hydroperoxide concentration by at least 20%, preferably 50% and in particular 90% within three minutes.

For the purpose of explaining the present invention, the expression “alkyl” comprises straight-chain and branched alkyl groups. These are preferably straight-chain or branched C₁-C₂₀-alkyl, more preferably C₁-C₁₂-alkyl, particularly preferably C₁-C₈-alkyl and very particularly preferably C₁-C₄-alkyl groups. Examples of alkyl groups are, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The expression “alkyl” also comprises substituted alkyl groups which generally have 1, 2, 3, 4 or 5, preferably 1, 2 or 3 and particularly preferably 1, substituents. These are preferably selected from alkoxy, cycloalkyl, aryl, hetaryl, hydroxyl, halogen, NE¹E², NE¹E²E³⁺, carboxylate and sulfonate. A preferred perfluoroalkyl group is trifluoromethyl.

For the purposes of the present invention, the expression “alkylene” is straight-chain or branched alkanediyl groups having 1 to 5 carbon atoms.

For the purposes of the present invention, the expression “cycloalkyl” comprises unsubstituted and also substituted cycloalkyl groups, preferably C₅-C₇-cycloalkyl groups, such as cyclopentyl, cyclohexyl or cycloheptyl. In the case of a substitution, these can generally carry 1, 2, 3, 4 or 5, preferably 1, 2 or 3 and particularly preferably 1, substituents. The substituents are preferably selected from alkyl, alkoxy, NE¹E², NE¹E²E³⁺, and halogen.

For the purposes of the present invention, the expression “heterocycloalkyl” comprises saturated, cycloaliphatic groups having generally 4 to 7, preferably 5 or 6, ring atoms in which 1 or 2 of the ring carbon atoms are replaced by heteroatoms selected from the elements oxygen, nitrogen and sulfur, and which may be optionally substituted, where, in the case of a substitution, these heterocycloaliphatic groups can carry 1, 2 or 3, preferably 1 or 2, particularly preferably 1, substituents. These substituents are preferably selected from alkyl, alkoxy, aryl, COOR^{o}, COO⁻M⁺, hydroxyl, halogen and NE¹E², particular preference being given to alkyl radicals. Examples of such heterocycloaliphatic groups which may be mentioned are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

For the purposes of the present invention, the expression “aryl” comprises unsubstituted and also substituted aryl groups and is preferably phenyl, tolyl, xylyl, mesityl, naphthyl, fluorenyl, anthracenyl, phenanthrenyl or naphthacenyl, particularly preferably phenyl or naphthyl, where these aryl groups, in the case of a substitution, can generally carry 1, 2, 3, 4 or 5, preferably 1, 2 or 3 and particularly preferably 1, substituents selected from the groups alkyl, alkoxy, carboxylate, trifluoromethyl, sulfonate, NE¹E², alkylene-NE¹E²; nitro, cyano or halogen. A preferred perfluoroaryl group is pentafluorophenyl.

For the purposes of the present invention, the expression “hetaryl” comprises unsubstituted or substituted heterocycloaromatic groups, preferably the groups furyl, thiophenyl, pyridyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl. These heterocycloaromatic groups can, in the case of a substitution, generally carry 1, 2 or 3 substituents selected from the groups alkyl, alkoxy, hydroxyl, carboxylate, sulfonate, NE¹E², alkylene-NE¹E² or halogen.

Within the scope of this invention, carboxylate and sulfonate are preferably a derivative of a carboxylic acid function or of a sulfonic acid function, in particular a metal carboxylate or sulfonate, a carboxylic acid ester or sulfonic acid ester function or a carboxamide or sulfonamide function. These include, for example, the esters with C₁-C₄-alkanols, such as methanol, ethanol, n-propanol, isopropanol, n-butanol, sec-butanol and tert-butanol.

The above explanations of the expressions “alkyl”, “cycloalkyl”, “aryl”, “heterocycloalkyl” and “hetaryl” apply correspondingly to the expressions “alkoxy”, “cycloalkoxy”, “aryloxy”, “heterocycloalkoxy” and “hetaryloxy”.

For the purposes of the present invention, the expression “acyl” is alkanoyl or aroyl groups having generally 2 to 11, preferably 2 to 8, carbon atoms, for example the formyl, acetyl, propanoyl, butanoyl, pentanoyl, hexanoyl, heptanoyl, 2-ethylhexanoyl, 2-propylheptanoyl, benzoyl or naphthoyl group.

The radicals E¹ to E⁶ are selected, independently of one another, from hydrogen, alkyl, cycloalkyl and aryl. The groups NE¹E² and NE⁴E⁵ are preferably N,N-dimethylamino, N,N-diethylamino, N,N-dipropylamino, N,N-diisopropylamino, N,N-di-n-butylamino, N,N-di-t-butylamino, N,N-dicyclohexylamino or N,N-diphenylamino.

Halogen is fluorine, chlorine, bromine or iodine, preferably fluorine, chlorine and bromine.

M⁺ is a cation equivalent, i.e. a monovalent cation or the fraction of a polyvalent cation corresponding to a positive single charge. The cation M⁺ serves merely as counterion for the neutralization of negatively charged substituent groups, such as the COO⁻ or the sulfonate group, and can in principle be selected arbitrarily. Preferably, therefore, alkali metal, in particular Na⁺, K⁺, Li⁺ ions or onium ions, such as ammonium, mono-, di-, tri-, tetraalkylammonium, phosphonium, tetraalkylphosphonium or tetraarylphosphonium ions, are used.

Corresponding statements apply to the anion equivalent X⁻, which serves merely as counterion of positively charged substituent groups, such as the ammonium groups, and can be chosen arbitrarily from monovalent anions and the fraction of a polyvalent anion corresponding to a negative single charge, where, in general, halide aions X⁻ are preferred, in particular chloride and bromide.

The values for x and y are an integer from 1 to 240, preferably an integer from 2 to 120.

Condensed ring systems may be aromatic, hydroaromatic and cyclic compounds linked (fused-on) by anellation. Condensed ring systems consist of two, three or more than three rings. Depending on the type of linkage, a distinction is made in the case of condensed ring systems between an ortho-anellation, i.e. each ring has in each case one edge, or two atoms, in common with each neighboring ring, and a peri-anellation, in which one carbon atom belongs to more than two rings. Among the condensed ring systems, preference is given to ortho-condensed ring systems. Preferred anellated rings are benzene, naphthalene and anthracene rings which, if desired, can carry one or more of the substituents specified above for aryl.

Preferably, the compounds of the formulae I.1 to I.3 used according to the invention are selected from compounds I.1a, I.1b, I.2a, I.2b and I.3a

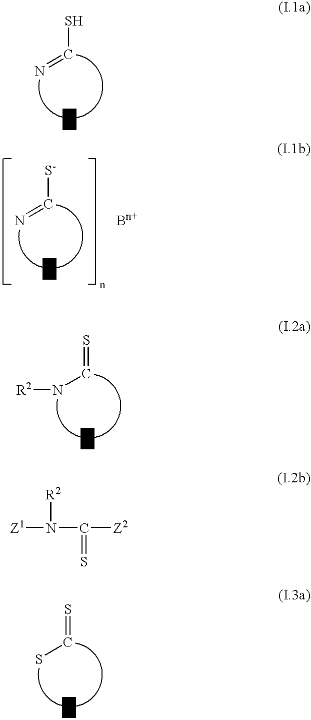

in which
Q is a divalent bridging group having 2 to 5 atoms between the flanking bonds,
Bⁿ⁺ is a mono-to trivalent cation,
n is an integer from 1 to 3
R² is hydrogen, alkyl, cycloalkyl, heterocycloalkyl, aryl, hetaryl, COOR^{a}, COO⁻M⁺, SO₃R^{a}, SO₃⁻M⁺, sulfonamide, NE¹E², (NE¹E²E³)⁺A⁻, OR^{a}, SR^{a}, (CHR^{b}CH₂O)_{y}R^{a}, (CH₂O)_{y}R^{a}, (CH₂CH₂NE¹)_{y}R^{a}, alkylaminocarbonyl, dialkylaminocarbonyl, alkylcarbonylamino or acyl, where
   R^{a} are in each case identical or different radicals selected from hydrogen, alkyl, cycloalkyl, aryl or hetaryl,
   E¹, E², E³ are in each case identical or different radicals selected from hydrogen, alkyl, cycloalkyl, aryl or hetaryl,
   R^{b} is hydrogen, methyl or ethyl,
   M⁺ is a cation equivalent,
   A⁻ is an anion equivalent and
   y is an integer from 1 to 250 which has the meanings given in claim 1

Z¹ and Z², independently of one another, are hydrogen, alkyl, cycloalkyl, heterocycloalkyl, aryl, heteroaryl, amino, aminocarbonyl, aminothiocarbonyl or thiocarbamidyl, where at least one of the radicals Z¹ or Z² is aryl or heteroaryl.

In particular, the compounds of the formula I.1 to I.3 are selected from

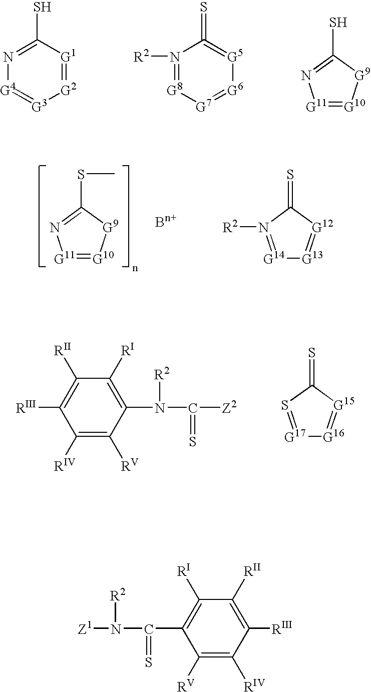

in which
G¹ is N or CR³,
G² is N or CR⁴,
G³ is N or CR⁵,
G⁴ is N or CR⁶,
   where
   R³, R⁴, R⁵ and R⁶, independently of one another, are hydrogen, alkyl, cycloalkyl, heterocycloalkyl, aryl, heteroaryl, COOR^{c}, COO⁻M⁺, SO₃R^{c}, SO₃⁻M⁺, sulfonamide, NE⁴E⁵, (NE⁴E⁵E⁶)⁺A⁻, OR^{c}, SR^{c}, (CHR^{d}CH₂O)_{w}R^{c}, (CH₂O)_{w}R^{c}, (CH₂CH₂NE⁴)_{w}R^{c}, alkylaminocarbonyl, dialkylaminocarbonyl, alkylcarbonylamino, halogen, nitro, acyl or cyano, where
   R^{c} are in each case identical or different radicals selected from hydrogen, alkyl, cycloalkyl, aryl or hetaryl,
   E⁴, E⁵, E⁶ are in each case identical or different radicals selected from hydrogen, alkyl, cycloalkyl, aryl or hetaryl,
   R^{d} is hydrogen, methyl or ethyl,
   M⁺ is a cation equivalent,
   A⁻ is an anion equivalent and
   w is an integer from 1 to 250,
      where two adjacent radicals R³ to R⁶ can also form an anellated ring system with one, two or three further rings,
G⁵ is BR⁷, CR⁷R⁸, SiR⁷R⁸, NR⁷, PR⁷, O or S,
G⁶ is BR⁹, CR⁹R¹⁰, SiR⁹R¹⁰, NR⁹, PR⁹, O or S,
G⁷ is BR¹¹, CR¹¹R¹², SiR¹¹R¹², NR¹¹, PR¹¹, O or S,
G⁸ is BR¹³, CR¹³R¹⁴, SiR¹³R¹⁴, NR¹³, PR¹³, O or S,
   where
   R⁷, R⁸, R⁹, R¹⁰, R¹¹, R¹², R¹³ and R¹⁴, independently of one another, can have the meanings given above for the radicals R³ to R⁶,
      where two geminal radicals R⁷R⁸, R⁹R¹⁰, R¹¹R¹² and/or R¹³R¹⁴ can also together be a double-bonded oxygen atom or sulfur atom,
      where two adjacent radicals R⁷, R⁹, R¹¹ and R¹³ can also be joined to anellated aliphatic or aromatic rings,
      where two adjacent radicals R⁷, R⁹, R¹¹ and R¹³ can also together be the bond fraction of a double bond between the atoms to which they are bonded,
G⁹ is BR¹⁵, CR¹⁵R¹⁶, SiR¹⁵R¹⁶, NR¹⁵, PR¹⁵, O or S,
G¹⁰ is N or CR¹⁷,
G¹¹ is N or CR¹⁸,
   where
   R¹⁵, R¹⁶, R¹⁷ and R¹⁸, independently of one another, can have the meanings given above for the radicals R³ to R⁶,
      where two adjacent radicals R¹⁷ and R¹⁸ can also form an anellated ring system with one, two or three further rings,
G¹² is BR¹⁹, CR¹⁹R²⁰, SiR¹⁹R²⁰, NR¹⁹, PR¹⁹, O or S,
G¹³ is BR²¹, CR²¹R²², SiR²¹R²², NR²¹, PR²¹, O or S,
G¹⁴ is BR²³, CR²³R²⁴, SiR²³R²⁴, NR²³, PR²³, O or S,
   where
   R¹⁹, R²⁰, R²¹, R²², R²³ and R²⁴, independently of one another, can have the meanings given above for the radicals R³ to R⁶,
      where two geminal radicals R¹⁹R²⁰, R²¹R²² and/or R²³R²⁴ can also together be a double-bonded oxygen atom or sulfur atom,
      where two adjacent radicals R¹⁹, R²¹ and R²³ can also be joined to anellated aliphatic or aromatic rings,
      where two adjacent radicals R¹⁹, R²¹ and R²³ can also together be the bond fraction of a double bond between the atoms to which they are bonded,
G¹⁵ is BR²⁵, CR²⁵R²⁶, SiR²⁵R²⁶, NR²⁵, PR²⁵, O or S,
G¹⁶ is BR²⁷, CR²⁷R²⁸, SiR²⁷R²⁹, NR²⁷, PR²⁷, or S,
G¹⁷ is BR²⁹, CR²⁹R³⁰, SiR²⁹R³⁰, NR²⁹, PR²⁹, O or S,
   where
   R²⁵, R²⁶, R²⁷, R²⁸, R²⁹ and R³⁰, independently of one another, can have the meanings given above for the radicals R³ to R⁶,
      where two geminal radicals R²⁵R²⁶, R²⁷R²⁸ and/or R²⁹R³⁰ can also together be a double-bonded oxygen atom or sulfur atom,
      where two adjacent radicals R²⁵, R²⁷ and R²⁹ can also be joined to anellated aliphatic or aromatic rings,
      where two adjacent radicals R²⁵, R²⁷ and R²⁹ can also together be the bond fraction of a double bond between the atoms to which they are bonded,
R^{I}, R^{II}, R^{III}, R^{IV} and R^{V}, independently of one another, are hydrogen, alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE⁷E⁸, alkylene-NE⁷E⁸, trifluoromethyl, nitro, alkoxycarbonyl, acyl or cyano, in which E⁷ and E⁸ are in each case identical or different radicals selected from hydrogen, alkyl, cycloalkyl and aryl, where two adjacent radicals R^{I} to R^{V}, together with the carbon atoms of the benzene ring to which they are bonded, can also be a condensed ring system having 1, 2 or 3 further rings, and
Bⁿ⁺, R², Z¹ and Z² are as defined above.

By way of example, mention may be made of the following compounds:

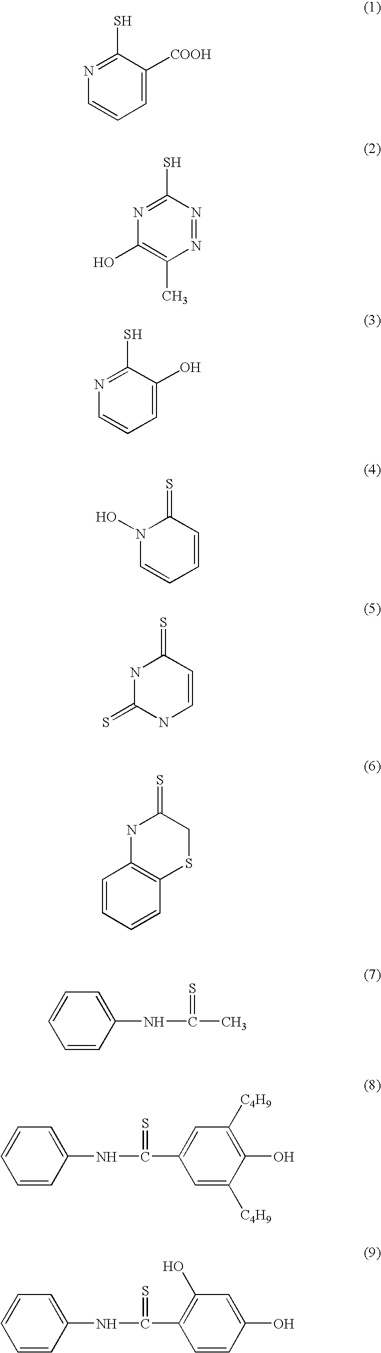

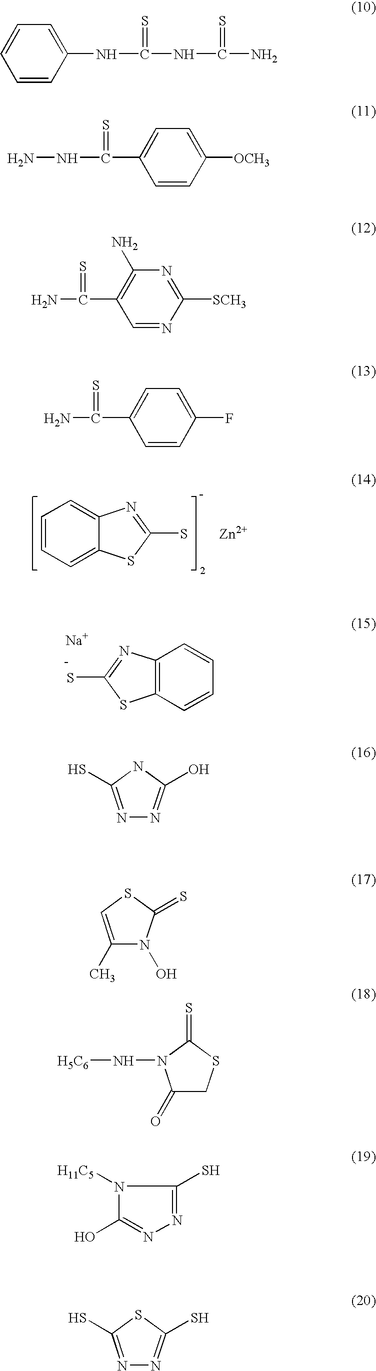

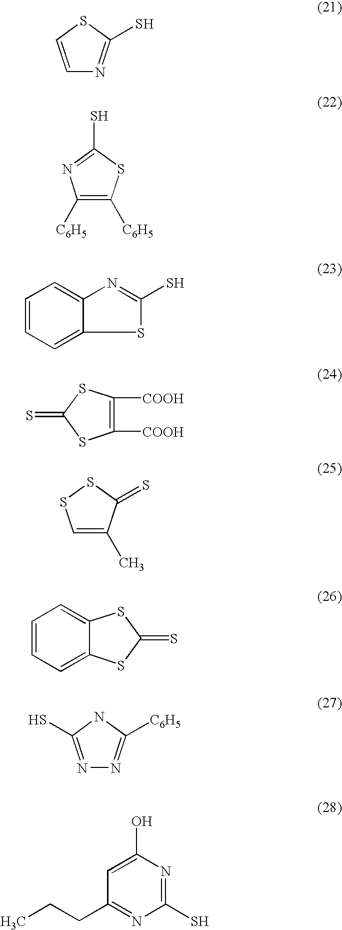

The compounds of the formulae I.1 to I.3 used according to the invention are advantageously suitable for avoiding or preventing damage to the skin by peroxides and/or hydroperoxides formed as a consequence of endogenous or exogenous factors. They are suitable for both the prevention and the treatment of damage to the skin of individuals, preferably mammals, in particular humans, useful animals or domestic animals. Use thereof can either be in cosmetic compositions such as bodycare compositions, decorative cosmetics etc., which are generally available without prescription, or else in dermatologicals, by which is understood medicaments for the therapy of skin disorders (dermatoses). Dermatologicals can additionally comprise at least one further active ingredient which is preferably selected from antimycotics, antiseptics, antibiotics, sulfonamides, disinfectants, corticoids, shale oil and tar sulfonates, astringents, antihydrotics, agents to combat acne, psoriasis, seborrhea and itching, keratolytics etc.

The treatment according to the invention of the affected skin can be directed toward individual disorders (anomalies, or medically abnormal states), although, if desired, it is also possible to treat two or more anomalies, if appropriate causally related to one another, using an agent according to the invention which comprises, if appropriate, an active ingredient combination.

Cosmetic and dermatological preparations based on compounds of the general formulae I.1 to I.3, as described above, and/or salts thereof offer effective protection against oxidative processes, against processes caused by radiation or reactive compounds, and against damage caused directly or indirectly by such processes. They are advantageously suitable for the treatment of cosmetic or dermatological changes in skin and hair, such as, for example, skin aging, loss of skin moisture, loss of skin elasticity, the formation of wrinkles or lines or of pigment disturbances or age spots.

The present invention further relates to the use of compounds of the general formulae I.1 to I.3 and/or salts thereof, if appropriate in combination with at least one further active ingredient and in preparations which comprise such active ingredients, for the cosmetic and dermatological treatment or prevention of undesired changes in the appearance of the skin, such as acne or greasy skin, keratoses, rosaceae and/or photosensitive, inflammatory, erythematous, allergic or autoimmune reactions, etc.

The compounds of the formulae I.1 to I.3 are particularly preferably suitable for rejuvenating and/or revitalizing the skin. Advantageously, energizing effects can also generally be observed. In particular, the compounds of the formulae I.1 to I.3 have a positive effect on the function of the mitochondria. Surprisingly, not only is oxidative damage to the skin prevented in this way, but existing damage can, at least partially, be repaired. In this connection, improvements, for example with regard to the moisture value and/or the elasticity of the skin, are noted. The use of compounds of the formulae I.1 to I.3 increases the further synthesis of collagen and/or elastin. This generally leads to a, at least partial, smoothing of wrinkles, and the complexion looks more radiant and fresher. Said advantageous effects are generally also accompanied by a positive subjective feeling of having overall “youthful” skin.

Formulations based on compounds of the general formulae I.1 to I.3 are also advantageously suitable for the treatment, care and cleansing of the skin and can serve as makeup product in cosmetics. They preferably comprise 0.001% by weight to 30% by weight of the active ingredient. In this connection, the composition is governed, for example, by the penetration properties of the active substance through the stratum corneum and its ability to form a depot in the skin.

According to a preferred embodiment, the use according to the invention of compounds of the formulae I.1 to I.3, derivatives and/or salts thereof, advantageously takes place by regular application, e.g. in the form of a cosmetic or dermatological preparation, over a period of time. This depends on the desired result, i.e. the period of time can extend over the lifetime of the user, preferably over a period of time up to three months, particularly preferably over a week to two months, if the aim is to build up a depot in the skin. For an aftersun application, the period of use for the purposes of the invention is a single application, but preferably a period of time of at least one day, particularly preferably over three days to three months, particularly preferably over one to two weeks.

For the purposes of the invention, it is advisable to topically apply the cosmetic or dermatological preparation of compounds of the formulae I.1 to I.3 in an amount of from 0.1 mg/cm² to 2 mg/cm², between once per week and 4 to 5 times daily, preferably 3 times per week to 3 times daily, particularly preferably once to twice daily. Active ingredient amounts and proportions are based on the active ingredient, meaning that for salts and derivatives, appropriate conversion is required.

Administration forms of compounds of the formulae I.1 to I.3 and if appropriate additional active ingredients that are intended for aftersun applications advantageously have penetration properties which permit rapid penetration of the substance into the skin. By contrast, for applications with a “preconditioning” character, rapid penetration is generally unimportant, whereas the ability to build up a depot in the skin is advantageous.

Surprisingly, in the case of the use according to the invention, effective treatment, but also prevention of prematurely aged skin (e.g. wrinkles, age spots, teleangiecstases, pigment disturbances), radiation-induced skin damage or radiation-induced negative changes in the skin, environmentally-induced (ozone, free radicals, singlet oxygen, reactive oxygen or nitrogen compounds, cigarette smoke, toxins) skin damage or environmentally-induced negative changes in the skin, photosensitive, inflammatory, erythematous, allergic or autoimmune changes in the skin (in particular acne, greasy or dry skin, keratoses, rosaceae, dertnatoses, atopic eczema, seborrheic eczema, photodermatoses, polymorphous photodermatoses), deficient, sensitive or hypoactive states of the skin and/or of the skin appendages, itching, dry skin states and horny layer barrier disturbances and/or hair loss and reduced hair growth is possible.

The use according to the invention of compounds of the formulae I.1 to I.3 in cosmetic and dermatological preparations also serves, however, in a surprising and unforeseeable manner, to calm sensitive and irritated skin, regulate collagen, hyaluronic acid, elastin synthesis, stimulate DNA synthesis, in particular in the case of deficient or hypoactive skin states, regulate the transcription and translation of matrix-destroying enzymes, in particular of matrix metalloproteinases, increase cell renewal and regeneration of the skin, increase endogenous protection and repair mechanisms for DNA, lipids and/or proteins, and, for pre- and post-treatment in the case of surgical interventions, to counteract skin irritations in particular and to promote regeneration processes of injured skin.

For use according to the invention, the cosmetic and dermatological preparations are applied to the skin in a sufficient amount in the manner customary for cosmetics.

For example, the compounds of the formulae I.1 to I.3 according to the invention, or salts thereof, are used in cosmetic compositions for cleansing the skin, such as bar soaps, toilet soaps, curd soaps, transparent soaps, luxury soaps, deodorizing soaps, cream soaps, baby soaps, skin protection soaps, abrasive soaps, syndets, liquid soaps, pasty soaps, soft soaps, washing pastes, liquid washing, showering and bath preparations, e.g. washing lotions, shower baths, shower gels, foam baths, cream foam baths, oil baths, bath extracts, scrub preparations, in-situ products, shaving foams, shaving lotions, shaving creams.

They are further suitable for skin cosmetic preparations such as W/O or O/W skin and body creams, day and night creams, eye creams, photoprotective compositions, aftersun products, handcare products, face creams, multiple emulsions, gelées, microemulsions liposome preparations, niosome preparations, antiwrinkle creams, face oils, lipogels, sport gels, moisturizing creams, bleaching creams, vitamin creams, skin lotions, care lotions, ampoules, aftershave lotions, preshaves, moisturizing lotions, tanning lotions, cellulite creams, depigmentation compositions, massage preparations, body powders, face tonics, face masks, deodorants, antiperspirants, nose strips, antiacne compositions, repellents, shaving compositions, hair removal compositions, personal hygiene compositions, footcare compositions, babycare compositions and others.

Furthermore, the compounds of the formulae I.1 to I.3 can be used in cosmetic compositions for haircare, such as hair treatments, hair lotions, hair rinses, hair emulsions, split-end fluids, neutralizing agents for permanent waves, hot-oil treatment preparations, conditioners, setting lotions, shampoos, hair tints and colorants, hairsprays, blow-waving lotions and setting compositions, shine sprays, hair brilliantines, hairstyling products, hair tonics, alopecia care compositions and others.

The compounds of the formulae I.1 to I.3 are also suitable for use in cosmetic preparations for decorative cosmetics, for example as makeup, powders, blushers, eyeshadows, kohl pencils, eyeliners, eye foundation cream, lipsticks, eyebrow pencils, contour pencils, concealing sticks, stage makeup, mascara, eyelash tinting and coloring, makeup removal products and others.

The cosmetic, hygiene, dermatological or pharmaceutical preparations can, depending on the field of use, be prepared as spray (pump spray or aerosol), foam, gels gel spray, lotion, cream, mousse, ointment, suspensions or powders.

It is also advantageous to administer the compounds of the formulae I.1 to I.3 or salts thereof if appropriate with further active ingredients in encapsulated form, e.g. as cellulose encapsulation, in gelatin, with polyamides, in niosomes, wax matrices, with cyclodextrins or liposomally encapsulated.

The preparations can comprise cosmetic auxiliaries as are customarily used in such preparations, e.g. preservatives, bactericides, perfumes, antifoams, dyes, pigments, thickeners, surface-active substances, emulsifiers, softening substances, reviving agents, fats, oils, waxes or other customary constituents of a cosmetic or dermatological formulation, such as alcohols, polyols, polymers, foam stabilizers, solubility promoters, electrolytes, organic acids, organic solvents or silicone derivatives.

In addition to the specified active ingredients, the preparations can comprise further compounds which have an antioxidative, free-radical scavenger, skin moisturizing or humectant, antierythematous, antiinflammatory or antiallergic action, in order to supplement or enhance the effect thereof. In particular, these compounds can be selected from the group of vitamins, plant extracts, α- and β-hydroxy acids, ceramides, antiinflammatory, antimicrobial or UV-filtering substances, and derivatives thereof and mixtures thereof.

The cosmetic or dermatological preparations generally comprise, based on the finished preparations, 0.0005 to 5% by weight, preferably 0.001 to 1% by weight, of at least one peroxide and/or hydroperoxide decomposer of the general formulae I.1 to I.3, as described above.

In a preferred embodiment, the peroxide decomposers used according to the invention are used in combination with at least one antioxidant that is effective as free-radical scavenger. The cosmetic or dermatological preparations then generally-comprise, based on the finished preparations, 0.0005 to 5% by weight, preferably 0.001 to 1% by weight, of at least one antioxidant that is effective as free-radical scavenger.

The additional antioxidants are generally compounds known per se. The antioxidants are advantageously selected from the groups of carotenoids, carotenes (e.g. α-carotene, β-carotene, lycopene) and derivatives thereof, chlorogenic acid and derivatives thereof, lipoic acid and derivatives thereof (e.g. dihydrolipoic acid), also (metal) chelating agents, EDTA, EGTA and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), butylhydroxytoluene, butylhydroxyanisole, and further antioxidants used customarily in cosmetic preparations.

In a particularly preferred embodiment, at least one compound of the general formulae I.1 to I.3, as defined above, is used in combination with at least one tocopherol or tocopherol derivative. The peroxide decomposers used for the combination are preferably selected from the compounds of the formulae (1) to (28). Specific embodiments are combinations of the compounds (1), (3), (4), (5), (6), (21), (27) and (28) with at least one tocopherol or tocopherol derivative, specifically with α-tocopherol (vitamin E).

Suitable tocopherols are very generally chroman-6-ols substituted in the 2-position by a 4,8,12-trimethyltridecyl radical, and derivatives thereof. Suitable derivatives are, for example, the esters with saturated and unsaturated, optionally substituted mono- and polycarboxylic acids, such as formic acid, acetic acid, propionic acid, butyric acid, oxalic acid, malonic acid, succinic acid, nicotinic acid, etc. These specifically include the acetates, succinates and nicotinates, specifically the acetates. Preferred tocopherols are α-, β-, γ-, δ- and ε-tocopherols and derivatives thereof. Particular preference is given to α-tocopherol, which is often equated with vitamin E, which is its main constituent and most effective constituent, and also α-tocopherol acetate.

The cosmetic and dermatological preparations generally comprise, based on the finished preparations, 0.00005 to 5% by weight, preferably 0.0001 to 1% by weight, in particular 0.0001 to 0.1% by weight, of at least one tocopherol or tocopherol derivative.

Preferably, the preparations furthermore comprise substances which absorb UV radiation in the UV-B and/or UV-A region. Suitable UV filters are, for example, 2,4,6-triaryl-1,3,5-triazines in which the aryl groups may in each case carry at least one substituent which is preferably selected from hydroxy, alkoxy, specifically methoxy, alkoxycarbonyl, specifically methoxycarbonyl and ethoxycarbonyl, and mixtures thereof. Also suitable are 4-aminobenzoic acid esters, where the amino group may be optionally alkylated or alkoxylated. These include, for example, isooctyl N,N-dimethyl-4-aminobenzoate.

Also suitable are cinnamic acid esters, benzophenones, camphor derivatives, and pigments which stop UV rays, such as titanium dioxide, talc and zinc oxide.

Also suitable are 2-hydroxybenzoic acid esters, such as, for example, the isooctyl ester. Further suitable UV filters are 2,4,6-trianilino(o-carbo-2′-ethylhexyl-1′-oxy)-1,3 5-triazine, 3-imidazol-4-ylacrylic acid and its ethyl ester, methyl o-aminobenzoate, glyceryl p-aminobenzoate, 2,2′-dihydroxy-4-methoxybenzophenone(dioxybenzone), 2-hydroxy-4-methoxy-4-methylbenzophenone(mexenone), triethanolamine salicylate, dimethoxyphenylglyoxalic acid, 3-(4′-sulfo)benzylidenebornan-2-one and its salts, 2,2′,4,4′-tetrahydroxybenzophenone, 2,2′-methylenebis[6(2H-benzotriazol-2-yl-4-(1,1,3,3-tetramethylbutyl)phenol], 2,2′-(1,4-phenylene)bis-1H-benzimidazole-4,6-disulfonic acid and its Na salt, 2,4-bis[4-(2-ethylhexyloxy)-2-hydroxy]phenyl-6-(4-methoxyphenyl)(1,3,5)triazine, 3-(4-methylbenzylidene)camphor, polyethoxyethyl 4-bis(polyethoxy)paraaminobenzoate, 2,4-dihydroxybenzophenone and/or 2,2′-dihydroxy-4,4′-dimethoxybenzophenone 5,5′-disodium sulfonate. Likewise advantageous is an aminosubstituted hydroxybenzophenone of the following formula:

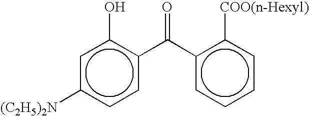

which is sold by BASF Aktiengesellschaft as UV-A filter under the trade name UVINUL® A Plus.

Also suitable are the following UV filter substances (the CAS No. (=acid) is given in each case in brackets): 4-amino acid (150-13-0), 3-(4′-trimethylammonium)benzylidenebornan-2 one methylsulfate (52793-97-2), 3,3,5-trimethylcyclohexyl salicylate(homosalate) (118-56-9), 2-hydroxy-4-methoxybenzophenone(oxybenzone) (131-57-7), 2-phenylbenzimidazole-5-sulfonic acid and its potassium, sodium and triethanolamine salts (27503-81-7), 3,3′-(1,4-phenylenedimethine)bis(7,7-dimethyl-2-oxabicyclo[2.2.1]heptane-1-methanesulfonic acid) and its salts (90457-82-2), polyethoxyethyl 4-bis(polyethoxy)aminobenzoate (113010-52-9), 2-ethylhexyl 4-dimethylaminobenzoate (21245-02-3), 2-ethylhexyl salicylate (118-60-5), 2-isoamyl 4-methoxycinnamate (71617-10-2), 2-ethylhexyl 4-methoxycinnamate (5466-77-3), 2-hydroxy-4-methoxybenzophenone-5-sulfonic acid (sulisobenzone) and the sodium salt (4065-45-6), 3-benzylidenebornan-2-one (16087-24-8), 1-(4′-isopropylphenyl)-3-phenylpropane-1,3-dione (63260-25-9), 4-isopropylbenzyl salicylate (94134-93-7), ethyl 2-cyano-3,3-diphenylacrylate (5232-99-5), 2′-ethylhexyl 2-cyano-3,3-diphenylacrylate (6197-30-4), menthyl o-aminobenzoate (134-09-8), 3,4-dimethoxyphenylglyoxal-acidic sodium (4732-70-1), 4-tert-butyl-4′-methoxydibenzoylmethane (70356-09-1), 2-(2H-benzotriazol-2-yl)-4-methyl-6-[2-methyl-3-[1,3,3,3-tetramethyl-1-[(trimethylsilyl)oxy]disiloxanyl]propyl]phenol (155633-54-8), 1,1-[(2,2′-dimethylpropoxy)carbonyl]-4,4-diphenyl-1,3-butadiene (363602-15-7).

The preparations can furthermore comprise inorganic pigments which stop UV rays and are based on metal oxides and/or other metal compounds which are insoluble or sparingly soluble in water, selected from the group of oxides of zinc (ZnO), titanium (TiO₂), iron (e.g. Fe₂O₃), zirconium (ZrO₂), silicon (SiO₂), manganese (e.g. MnO₂), aluminum (Al₂O₃), cerium (e.g. Ce₂O₃), mixed oxides of the corresponding metals, and mixtures of such oxides.

The inorganic pigments here may be present in coated form, i.e. have been surface-treated. This surface treatment can, for example, consist in providing the pigments with a thin hydrophobic layer by a method known per se, as described in DE-A-33 14742.

The list of specified UV filters which can be used in combination with the active ingredient combinations according to the invention is not of course intended to be limiting.

The total amount of the filter substances is generally 0.1% by weight to 30% by weight, preferably 0.5 to 15% by weight, in particular 1 to 10% by weight, based on the total weight of the preparations, in order to provide cosmetic preparations which protect the skin from the entire range of ultraviolet radiation.

If a lipid phase is used, then this is preferably selected from mineral oils, mineral waxes, branched and/or unbranched hydrocarbons and hydrocarbon waxes, triglycerides of saturated and/or unsaturated, branched and/or unbranched C₈-C₂₄-alkanecarboxylic acids; synthetic, semisynthetic or natural oils, such as olive oil, palm oil, almond oil or mixtures; oils, fats or waxes; esters of saturated and/or unsaturated, branched and/or unbranched C₃-C₃₀-alkanecarboxylic acids and saturated and/or unsaturated, branched, and/or unbranched C₃-C₃₀-alcohols, esters of aromatic carboxylic acids and saturated and/or unsaturated, branched and/or unbranched C₃-C₃₀ alcohols, for example isopropyl myristate, isopropyl stearate, hexyldecyl stearate, oleyl oleate; synthetic, semisynthetic and natural mixtures of the abovementioned esters, such as jojoba oil, alkyl benzoates or silicone oils, such as, for example, cyclomethicone, dimethylpolysiloxane, diethylpolysiloxane, octamethylcyclotetrasiloxane, and mixtures thereof, or dialkyl ethers.

If an aqueous phase is used, then it additionally comprises, if appropriate, a water-miscible solvent, such as C₁-C₁₀-, preferably C₁-C₅-alcohols, -diols, or -polyols, and ethers thereof, preferably ethanol, isopropanol, propylene glycol, glycerol, ethylene glycol monoethyl ether etc.

Suitable emulsifiers are preferably known W/O and O/W emulsifiers, such as polyglycerol esters, sorbitan esters or partially esterified glycerides.

To be mentioned as suitable solubility promoters are, in particular, ethoxylated sorbitan esters, ethoxylated lanolin alcohols and ethoxylated castor oil.

Customary native and synthetic thickeners and gel formers in the formulations are crosslinked polyacrylic acids and derivatives thereof, polysaccharides such as xanthan gum or alginates, carboxymethylcellulose or hydroxycarboxymethylcellulose, hydrocolloids, such as gum arabic or montmorillonite minerals, such as bentonites or fatty alcohols, polyvinyl alcohol and polyvinylpyrrolidone.

Suitable propellants for aerosols are the customary propellants, for example propane, butane, pentane, dimethyl ether and others.

The invention further provides a cosmetic or pharmaceutical agent for treating skin for avoiding or reducing damage to the skin by peroxides and/or hydroperoxides formed as a result of endogenous or exogenous factors, comprising:

I) at least one peroxide decomposer, as defined above,

II) if appropriate at least one antioxidant that is effective as free-radical scavenger, and

III) at least one cosmetically or pharmaceutically acceptable carrier.

The agents according to the invention are, in particular, not deodorants, antiperspirants, agents for inhibiting or preventing pigment production, agents for the treatment of fibrotic disorders and agents promoting hair growth.

Preferably, the agents comprise component 1) in an amount of from 0.001 to 30% by weight, in particular 0.01 to 25% by weight, based, on the total weight of the agent.

As regards suitable components II) and III), reference is made to that stated above. A preferred embodiment covers agents which comprise at least one tocopherol or tocopherol derivative, as defined above.

The formulation basis of pharmaceutical formulations according to the invention preferably comprises pharmaceutically acceptable auxiliaries. Of pharmaceutical acceptability are the auxiliaries known for use in the field of pharmacy, food technology and related fields, in particular the auxiliaries listed in the relevant pharmacopoeia (e.g. DAB Ph. Eur. BP NF), and other auxiliaries whose properties do not preclude a physiological application.

Suitable auxiliaries may be: glidants, wetting agents, emulsifying and suspending agents, preservatives, antioxidants, antiirritative substances, chelating agents, emulsion stabilizers, film formers, gel formers; odor masking agents, resins, hydrocolloids, solvents, solubility promoters, neutralizing agents, permeation accelerators, pigments, quaternary ammonium compounds, refatting and superfatting agents, ointment, cream or oil bases, silicone derivatives, stabilizers, sterilizing agents, propellants, drying agents, opacifiers, thickeners, waxes, softeners, white oils. Formulation in this regard is based on expert knowledge as given, for example, in Fiedler, H.P. Lexikon der Hilfsstoffe für Pharmazie, Kosmetik und angrenzende

Gebiete [Lexicon of auxiliaries for pharmacy, cosmetics and related fields], 4th Edition, Aulendorf: ECV-Editio-Kantor-Verlag, 1996.

Examples of suitable dermatological formulations are ointments, creams, hydrogels, pastes or plasters, and also liquid drug forms, such as solutions, emulsions, in particular-oil-in-water emulsions, suspensions, for example lotions etc. If desired, liposomes or microspheres can also be used.

According to the invention, the formulations are administered transdermally (topically).

To prepare the dermatological agents according to the invention, the active ingredients can be mixed or diluted with a suitable auxiliary (excipient). Excipients may be solid, semisolid or liquid materials which can serve as vehicles, carriers or medium for the active ingredient. Further auxiliaries can be admixed if desired in the manner known to the person skilled in the art.

If desired, two or more active ingredient components can be formulated together. However, they can also be firstly processed separately and then be combined in a suitable drug form.

The present invention further provides agents in the form of a commercial pack with at least one agent based on

i) at least one compound of the general formulae I.1 to. I.3, as defined above,

ii) if appropriate at least one further active ingredient, and

iii) a formulation base;

if appropriate together with instructions for the therapeutic use of the compounds of the general formulae I.1 to I.3.

It goes without saying that commercial packs according to the invention can also comprise further preparations, in particular formulations containing active ingredients, as well as comprehensive instructions extending beyond the content specified above.

Preferably, the cosmetic and dermatological agents are in the form of emulsions, in particular in the form of water-in-oil (W/O) or oil-in-water (O/W) emulsions.

However, it is also possible to choose other types of formulation, for example gels, oils, oleogels, multiple emulsions, for example in the form of W/O/W or O/W/O emulsions, anhydrous ointments or ointment bases, etc. Emulsifier-free formulations, such as hydrodispersions, hydrogels or a Pickering emulsion are also advantageous embodiments.

The preparation of emulsions takes place by known methods. Besides at least one peroxide decomposer I.1-I.3, the emulsions generally comprise customary constituents, such as fatty alcohols, fatty acid esters and in particular fatty acid triglycerides, fatty acids, lanolin and derivatives thereof, natural or synthetic oils or waxes and emulsifiers in the presence of water. The selection of additives specific to the type of emulsion and the preparation of suitable emulsions is described, for example, in Schrader, Grundlagen und Rezepturen der Kosmetika [Fundamentals and formulations of cosmetics], Hüthig Buch Verlag, Heidelberg, 2nd Edition, 1989, third part, to which reference is hereby expressly made.

A suitable emulsion as W/O emulsion, e.g. for a skin cream etc., generally comprises an aqueous phase which is emulsified in an oil or fat phase using a suitable emulsifier system. For the provision of the aqueous phase, a polyelectrolyte complex can be used.

The invention is explained in more detail by reference to the following, nonlimiting examples.

### EXAMPLE

General procedure for determining the synergistic effect of peroxide decomposers used according to the invention with tocopherol

3 ml of a 2% strength solution of azobis(2,4.dimethylvaleronitrile). (V65, Wako Chemical Co.) in squalene are poured together with 250 ppm of test substance (based on the squalene solution) into Fiolax test tubes (12×100 ml) and thoroughly mixed using a laboratory shaker. The samples are then stored open in a circulatory drying cabinet for 30 days at 40° C. At intervals of 7 days, the squalene content of the samples is ascertained using gas chromatography. The reference used is a sample without the free-radical initiator. The data points are documented in an XY graph and the effectiveness of the test substances is determined by reference to the slope of the decomposition curves in comparison to the reference sample.

A measure of the protective effect of a test compound is the factor QS, which is a measure of how much a stabilizer reduces the degradation rate. QSₓ=degradation rate₀/degradation rateₓ

The results are given in table 1 below:

| | | | QS + |
|---|---|---|---|
| | | | 250 ppm |
| Ex. No. | Compound | QS | Tocopherol |
| 1 | Reference | 1.0 | — |
| (comparison) | | | |
| 2 | Tocopherol | 1.3 | — |
| (comparison) | | | |
| 3 | 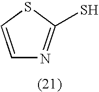 | 2.75 | 4.25 |
| 4 | 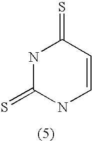 | 1.13 | 2.08 |
| 5 | 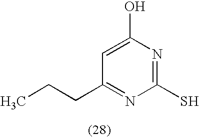 | 2.58 | 4.44 |
| 6 | 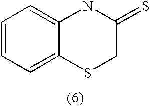 | 3.03 | 3.75 |
| 7 | 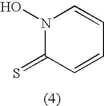 | 1.47 | 2.58 |
| 8 | 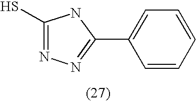 | 1.34 | 1.8 |
| 9 | 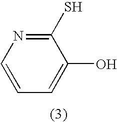 | 1.66 | 2.25 |
| 10 | 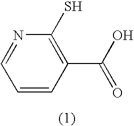 | 1.18 | 1.65 |

The compounds according to the invention even on their own exhibit good effect as peroxide decomposers, which can again be significantly increased through the addition of tocopherol.

Described below are dermocosmetic preparations according to the invention comprising peroxide decomposers according to compound I.1-I.3, for example compounds which are given in table 1.

### Example 11

### Use of the Peroxide Decomposers in a Protective Day Cream—O/W TypeAI 1%

| | % | Ingredient (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 68.6 | Demineralized water |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | Compound 21 |
| E | q.s. | Sodium Hydroxide |

AI 5%:

| | % | Ingredients (INCI) |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 64.6 | Demineralized water |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | Compound 21 |
| E | q.s. | Sodium Hydroxide |

Preparation: Heat phases A and B separately from one another to about 80° C. Stir phase B into phase A and homogenize. Incorporate phase C into the combined phases A and B and homogenize. Cool to about 40° C. with stirring. Add phase D, adjust the pH to about 6.5 with phase E and homogenize. Cool to room temperature with stirring.

### Example 12

### Use of the Peroxide Decomposers in a Face Cleansing Lotion—O/W Type AI 1%

| | % | Ingredients (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume Oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Compound 5 |
| | 60.7 | Demineralized water |

AI 5%:

| | % | Ingredients (INCI) |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume Oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Compound 5 |
| | 56.7 | Demineralized water |

Preparation: Dissolve phase A. Stir phase B. into phase A, incorporate phase C into the combined phases A and B. Dissolve phase D, stir into the combined phases A, B and C and homogenize. After stir for 15 minutes.

### Example 13

### Use of the Peroxide Decomposers in a Daily Care Body Spray

### AI 1%:

| | % | Ingredients (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Compound 28 |
| | 59.2 | Alcohol |

AI 5%:

| | % | Ingredients (INCI) |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Compound 28 |
| | 55.2 | Alcohol |

Preparation: Weigh in the components of phase A and dissolve to give a clear solution.

### Example 14

### Use of the Peroxide Decomposers in a Skincare Gel AI 1%

| | % | Ingredients (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume Oil |
| B | 3.0 | Panthenol |
| | 0.6 | Carbomer |
| | 1.0 | Compound 6 |
| | 75.4 | Aqua dem. |
| C | 0.8 | Triethanolamine |

AI 5%:

| | % | Ingredients (INCI) |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume Oil |
| B | 3.0 | Panthenol |
| | 0.6 | Carbomer |
| | 5.0 | Compound 6 |
| | 71.4 | Aqua dem. |
| C | 0.8 | Triethanolamine |

Preparation: Dissolve phase A to give a clear solution. Allow phase B to swell and neutralize with phase C. Stir phase A into the homogenized phase B and homogenize.

### Example 15

### Use of the Peroxide Decomposers in an Aftershave Lotion AI 1%

| | % | Ingredients (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume Oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 1.0 | Compound 4 |
| | 0.1 | Triethanolamine |
| | 63.5 | Demineralized water |

AI 5%:

| | % | Ingredients (INCI) |
|---|---|---|
| A | 10.0 | Cetearyl Ethylhexanoate |
| | 5.0 | Tocopheryl Acetate |
| | 1.0 | Bisabolol |
| | 0.1 | Perfume Oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 5.0 | Compound 4 |
| | 0.1 | Triethanolamine |
| | 59.5 | Demineralized water |

Preparation: Mix the components of phase A. Dissolve phase B, incorporate into phase A and homogenize.

### Example 16

### Use of the Peroxide Decomposers in an Aftersun Lotion AI 1%

| | % | Ingredients (INCI) |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume Oil |
| B | 1.0 | Panthenol |
| | 15.0 | Alcohol |
| | 3.0 | Glycerin |
| | 1.0 | Compound 27 |
| | 63.2 | Aqua dem. |
| C | 0.2 | Triethanolamine |

AI 5%:

| | % | Ingredients (INCI) |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume Oil |
| B | 1.0 | Panthenol |
| | 15.0 | Alcohol |
| | 3.0 | Glycerin |
| | 5.0 | Compound 27 |
| | 59.2 | Aqua dem. |
| C | 0.2 | Triethanolamine |

Preparation: Mix the components of phase A. Stir phase B into phase A with homogenization. Neutralize with phase C and homogenize again.

### Example 17

### Use of the Peroxide Decomposers in a Sunscreen Lotion AI 1%

| | % | Ingredients (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C₁₂₋₁₃ Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 59.7 | Demineralized water |
| D | 1.0 | Compound 3 |
| | 1.0 | Phenoxyethanol, Methylparaben, Ethylparaben, Butylparaben, |
| | | Propylparaben, Isobutylparaben |
| | 0.3 | Bisabolol |

AI 5%:

| | % | Ingredients (INCI) |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C₁₂₋₁₃ Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C₁₂₋₁₃ Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 55.7 | Demineralized water |
| D | 5.0 | Compound 3 |
| | 1.0 | Phenoxyethanol, Methylparaben, Ethylparaben, Butylparaben, |
| | | Propylparaben, Isobutylparaben |
| | 0.3 | Bisabolol |

Preparation: Heat the components of phases A and B separately from one another to about 80° C. Stir phase B into phase A and homogenize. Heat phase C to about 80° C. and stir into the combined phases A and B with homogenization. Cool to about 40° C. with stirring, add phase D and homogenize again.

### Example 18

### Use of the Peroxide Decomposers in a Sunscreen Lotion—O/W Type AI 1%

| | % | Ingredients (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, |
| | | Squalane, Polysorbate 60 |
| | 0.2 | Disodium EDTA |
| | 5.0 | Propylene Glycol |
| | 0.5 | Panthenol |
| | 60.9 | Demineralized water |
| D | 1.0 | Compound 1 |
| | 0.5 | Phenoxyethanol, Methylparaben, Butylparaben, Ethylparaben, |
| | | Propylparaben, Isopropylparaben |
| | 1.0 | Tocopheryl Acetate |
| | 0.2 | Bisabolol |

AI 5%:

| | % | Ingredients (INCI) |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, |
| | | Squalane, Polysorbate 60 |
| | 0.2 | Disodium EDTA |
| | 5.0 | Propylene Glycol |
| | 0.5 | Panthenol |
| | 56.9 | Demineralized water |
| D | 5.0 | Compound 1 |
| | 0.5 | Phenoxyethanol, Methylparaben, Butylparaben, Ethylparaben, |
| | | Propylparaben, Isopropylparaben |
| | 1.0 | Tocopheryl Acetate |
| | 0.2 | Bisabolol |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 19

### (Use of the Peroxide Decomposers in a Sunscreen Lotion—O/W Type AI 1%

| | % | Ingredients (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Bees Wax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Demineralized water |
| D | 1.0 | Compound 19 |
| | 1.0 | Tocopheryl Acetate |
| | 0.2 | Bisabolol |
| | q.s. | Perfume Oil |
| | q.s. | Preservative |

AI 5%:

| | % | Ingredients (INCI) |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Bees Wax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Demineralized water |
| D | 5.0 | Compound 19 |
| | 1.0 | Tocopheryl Acetate |
| | 0.2 | Bisabolol |
| | q.s. | Perfume Oil |
| | q.s. | Preservative |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 20

### Use of the Peroxide Decomposers in a Foot Balm AI 1%

| | % | Ingredients (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Demineralized water |
| | q.s. | Preservative |
| C | 1.0 | Bisabolol |
| | 1.0 | Tocopheryl Acetate |
| D | 1.0 | Compound 7 |
| | 5.0 | Witch Hazel Extract |

AI 5%:

| | % | Ingredients (INCI) |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Demineralized water |
| | q.s. | Preservative |
| C | 1.0 | Bisabolol |
| | 1.0 | Tocopheryl Acetate |
| D | 5.0 | Compound 7 |
| | 5.0 | Witch Hazel Extract |

Preparation: Heat the components of phases A and B separately from one another to about 80° C. Stir phase B into phase A with homogenization. Cool with stirring to about 40° C., add phases C and D and briefly after homogenize. Cool to room temperature with stirring.

### Example 21

### Use of the Peroxide Decomposers in a W/O Emulsion with Bisabolol AI 1%

| | % | Ingredients (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Demineralized water |
| C | 1.0 | Compound 15 |
| | 0.5 | Tocopheryl Acetate |
| | 0.6 | Bisabolol |

AI 5%:

| | % | Ingredients (INCI) |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Demineralized water |
| C | 5.0 | Compound 15 |
| | 0.5 | Tocopheryl Acetate |

Preparation: Heat phases A and B separately from one another to about 85° C. Stir phase B into phase A and homogenize. With stirring, cool to about 40° C., add phase C and briefly homogenize again. Cool to room temperature with stirring.

### Example 22

Described below are dermocosmetic preparations according to the invention comprising peroxide decomposers according to compound I.1-I.3, for example compounds which are specified in table 1. The specified compounds are referred to below as peroxide decomposers and the amount used is given in % by weight.

Clear shampoo

| Ingredients (INCI) | 1 | 2 | 3 | 4 | 5 |
|---|---|---|---|---|---|
| Sodium Laureth Sulfate | 13.00 | 15.00 | 10.50 | 12.50 | 10.00 |
| Codamidopropyl Betaine | 7.50 | 7.00 | 5.00 | 5.50 | 10.00 |
| PEG-7 Glyceryl Cocoate | 2.00 | 2.50 | 3.50 | 5.00 | 2.30 |
| Perfume Oil | 0.10 | 0.10 | 0.10 | 0.10 | 0.10 |
| Peroxide decomposer | 1.0 | 5.0 | 0.1 | 0.5 | 10.0 |
| D-Panthenol USP | 1.00 | 1.50 | 1.80 | 1.70 | 1.40 |
| Preservative | 0.10 | 0.10 | 0.10 | 0.10 | 0.10 |
| Citric Acid | 0.10 | 0.10 | 0.10 | 0.10 | 0.10 |
| Luviquat ® Ultra Care | 1.50 | 1.00 | 1.50 | 1.20 | 1.10 |
| Sodium Chloride | 1.50 | 1.40 | 1.40 | 1.30 | 1.50 |
| Demineralized water | ad 100 | ad 100 | ad 100 | ad 100 | ad 100 |

Shampoo

| Ingredients (INCI) | 1 | 2 | 3 | 4 | 5 |
|---|---|---|---|---|---|
| Sodium Laureth Sulfate | 35.00 | 40.00 | 30.00 | 45.00 | 27.00 |
| Decyl Glucoside | 5.00 | 5.50 | 4.90 | 3.50 | 7.00 |
| Cocamidopropyl Betaine | 10.00 | 5.00 | 12.50 | 7.50 | 15.00 |
| Perfume Oil | 0.10 | 0.10 | 0.10 | 0.10 | 0.10 |
| Peroxide decomposer | 1.0 | 5.0 | 0.1 | 0.5 | 10.0 |
| D-Panthenol USP | 0.50 | 1.00 | 0.80 | 1.50 | 0.50 |
| Preservative | 0.10 | 0.10 | 0.10 | 0.10 | 0.10 |
| Citric Acid | 0.10 | 0.10 | 0.10 | 0.10 | 0.10 |
| Laureth-3 | 0.50 | 2.00 | 0.50 | 0.50 | 2.00 |
| Sodium Chloride | 1.50 | 1.50 | 1.50 | 1.50 | 1.50 |
| Demineralized water | ad 100 | ad 100 | ad 100 | ad 100 | ad 100 |

Clear conditioner shampoo

| Ingredients (INCI) | 1 | 2 | 3 | 4 | 5 |
|---|---|---|---|---|---|
| ® Disodium | 10.00 | 15.00 | 20.00 | 12.00 | 17.00 |
| Cocoamphodiacetate | | | | | |
| ® Decyl Glucoside | 5.00 | 6.00 | 7.00 | 8.00 | 4.00 |
| ® Cocamidopropyl | 15.00 | 12.00 | 10.00 | 18.00 | 20.00 |
| Betaine | | | | | |
| Luviquat ® FC 550 | 0.30 | 0.20 | 0.20 | 0.20 | 0.30 |
| Perfume Oil | 0.10 | 0.10 | 0.10 | 0.10 | 0.10 |
| Peroxide decomposer | 20.0 | 5.0 | 1.0 | 0.5 | 10.0 |
| Cremophor ® PS 20 | 5.00 | 1.00 | 1.00 | 7.00 | 5.00 |
| Preservative | 0.10 | 0.10 | 0.10 | 0.10 | 0.10 |
| ® Laureth-3 | 2.00 | 1.00 | 0.50 | 2.00 | 2.00 |
| Citric Acid | 0.20 | 0.20 | 0.20 | 0.20 | 0.20 |
| PEG-12 Distearate | 3.00 | 2.00 | 2.00 | 3.00 | 2.50 |
| Demineralized water | ad 100 | ad 100 | ad 100 | ad 100 | ad 100 |

Foam O/W emulsions

| | Emulsion 1 | % by | Emulsion 2 | % by |
|---|---|---|---|---|
| | % by wt. | vol. | % by wt. | vol. |
| Stearic Acid | 5.00 | | 1.00 | |
| Cetyl Alcohol | 5.50 | | | |
| Cetearyl Alcohol | | | 2.00 | |
| PEG-40 Stearate | 8.50 | | | |
| PEG-20 Stearate | | | 1.00 | |
| Caprylic/Capric Triglyceride | 4.00 | | 2.00 | |
| C₁₂₋₁₅ Alkyl Benzoate | 10.00 | | 15.00 | |
| Cyclomethicone | 4.00 | | | |
| Dimethicone | | | 0.50 | |
| Peroxide decomposer | 5.0 | | 10.0 | |
| Ethylhexyl Isostearate | | | 5.00 | |
| Myristyl Myristate | | | 2.00 | |
| Ceresin | 1.50 | | | |
| Glycerin | | | 3.00 | |
| Hydroxypropyl Starch | 1.00 | | 3.50 | |
| Phosphate | | | | |
| BHT | | | 0.02 | |
| Disodium EDTA | 0.50 | | 0.10 | |
| Perfume oil, Preservative | q.s. | | q.s. | |
| Colorant | q.s. | | q.s. | |
| Potassium Hydroxide | q.s. | | q.s. | |
| Demineralized water | ad 100 | | ad 100 | |
| | adjust pH | | adjust pH | |
| | to 6.5-7.5 | | to 5.0-6.0 | |
| Emulsion 1 | | 70 | | |
| Emulsion 2 | | | | 35 |
| Nitrogen | | 30 | | |
| Propane/Butane | | | | 65 |

Conditioner shampoo with pearlescence

| | 1 | 2 | 3 |
|---|---|---|---|
| Polyquaternium-10 | 0.50 | 0.50 | 0.40 |
| Sodium Laureth Sulfate | 9.00 | 8.50 | 8.90 |
| Codamidopropyl Betaine | 2.50 | 2.60 | 3.00 |
| Uvinul ® MS 40 | 1.50 | 0.50 | 1.00 |
| Peroxide decomposer | 1.0 | 5.0 | 0.5 |
| Pearlescent solution | 2.00 | 2.50 | |
| Disodium EDTA | 0.10 | 0.15 | 0.05 |
| Preservative, perfume oil, thickener | q.s. | q.s. | q.s. |
| Demineralized water | ad 100 | ad 100 | ad 100 |

Adjust pH to 6.0

Clear conditioner shampoo

| | 1 | 2 | 3 |
|---|---|---|---|
| Polyquaternium-10 | 0.50 | 0.50 | 0.50 |
| Sodium Laureth Sulfate | 9.00 | 8.50 | 9.50 |
| Peroxide decomposer | 5.0 | 0.1 | 3.0 |
| Uvinul M ® 40 | 1.00 | 1.50 | 0.50 |
| | 0.20 | 0.20 | 0.80 |
| Preservative, perfume oil, thickener | q.s. | q.s. | q.s. |
| Demineralized water | ad 100 | ad 100 | ad 100 |

Adjust pH to 6.0

Clear conditioner shampoo with volume
effect

| | 1 | 2 | 3 |
|---|---|---|---|
| Sodium Laureth Sulfate | 10.00 | 10.50 | 11.00 |
| Uvinul ® MC 80 | 2.00 | 1.50 | 2.30 |
| Peroxide decomposer | 10.0 | 0.1 | 0.5 |
| Cocamidopropyl Betaine | 2.50 | 2.60 | 2.20 |
| Disodium EDTA | 0.01 | 0.10 | 0.01 |
| Preservative, Perfume Oil, Thickener | q.s. | q.s. | q.s. |
| Demineralized Water | ad 100 | ad 100 | ad 100 |

Adjust pH to 6.0

Gel cream

| | 1 | 2 | 3 | 4 |
|---|---|---|---|---|
| Acrylates/C10-30 Alkyl Acrylate | 0.40 | 0.35 | 0.40 | 0.35 |
| Crosspolymer | | | | |
| Carbomer | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus ™ | 2.00 | 1.50 | 0.75 | 1.00 |
| UvaSorb ® k2A | | 3.00 | | |
| Ethylhexyl | | | | |
| Bis-Isopentylbenzoxazolylphenyl | | | | |
| Melamine | | | | |
| Uvinul ® MC 80 | 3.00 | | 1.00 | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Uvinul ® T 150 | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | 3.50 | 4.00 | 7.00 | 9.00 |
| Titanium Dioxide - microfine | 1.00 | | 3.00 | |
| Zinc Oxide - microfine | | | | 0.25 |
| Peroxide decomposer | 0.1 | 0.5 | 1.0 | 0.02 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume Oil | 0.20 | | 0.20 | |
| Demineralized Water | ad 100 | ad 100 | ad 100 | ad 100 |

Adjust pH to 6.0

OW Sunscreen formulation

| | 1 | 2 | 3 | 4 | 5 | 6 | 7 |
|---|---|---|---|---|---|---|---|
| Glyceryl Stearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Peroxide decomposer | 10.0 | 0.5 | 3.0 | 5.0 | 0.1 | 0.02 | 7.5 |
| Uvinul ® A Plus ™ | 2.00 | 1.50 | 0.75 | 1.00 | 2.10 | 4.50 | 5.00 |
| UvaSorb ® k2A | | | | | | | |
| Ethylhexyl Bis- | | | | | | | |
| Isopentylbenzoxazolylphenyl | | | | | | | |
| Melamine | | | | | | | |
| Ethylhexyl Methoxycinnamate | | | | | 5.00 | 6.00 | 8.00 |
| Uvinul ® MC 80 | | | | | | | |
| Bis-Ethylhexyloxyphenol | | 1.50 | | 2.00 | 2.50 | | 2.50 |
| Methoxyphenyl Triazine | | | | | | | |
| Butyl | | | 2.00 | | 2.00 | 1.50 | |
| Methoxydibenzoylmethane | | | | | | | |
| Disodium Phenyl | 2.50 | | 0.50 | 2.00 | | 0.30 | |
| Dibenzimidazole Tetrasulfonate | | | | | | | |
| Ethyhexyl Triazone Uvinul ® T | 4.00 | | 3.00 | 4.00 | | 2.00 | |
| 150 | | | | | | | |
| Octocrylene | | 4.00 | | | | | 7.50 |
| Diethylhexyl Butamido Triazone | 1.00 | | | 2.00 | 1.00 | | 1.00 |
| Phenylbenzimidazole Sulfonic | 0.50 | | 3.00 | | | | |
| Acid | | | | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide - microfine | 1.00 | | 3.00 | | 3.50 | | 1.50 |
| Zinc Oxide - microfine | | | | 0.25 | | 2.00 | |
| C₁₂₋₁₅ Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium Iminodisuccinate | | | | 0.25 | 1.00 | | |
| Ethanol | 2.00 | 1.50 | | 3.00 | | 1.20 | 5.00 |
| Perfume Oil | 0.10 | 0.25 | 0.30 | | 0.40 | 0.20 | |
| Demineralized Water | ad | ad | ad | ad | ad | ad 100 | ad |
| | 100 | 100 | 100 | 100 | 100 | | 100 |

Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Sodium Carbomer | | 0.20 | | 0.30 | |
| Acrylates/C₁₀₋₃₀ Alkyl Acrylate | 0.50 | | 0.40 | 0.10 | 0.50 |
| Crosspolymer | | | | | |
| Xanthan Gum | | 0.30 | 0.15 | | |
| Peroxide decomposer | 5.0 | 0.5 | 3.0 | 0.1 | 10.0 |
| Uvinul ® A Plus ™ | 2.00 | 1.50 | 0.75 | 1.00 | 2.10 |
| UvaSorb ® k2A Ethylhexyl Bis- | | 3.50 | | | |
| Isopentylbenzoxazolylphenyl Melamine | | | | | |
| Ethylhexyl Methoxycinnamate Uvinul ® | | | | | 5.00 |
| MC 80 | | | | | |
| Bis-Ethylhexyloxyphenol | | 1.50 | | 2.00 | 2.50 |
| Methoxyphenyl Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic | | 1.50 | | 1.00 | 1.00 |
| Acid | | | | | |
| Diethylhexyl 2,6-Naphthalate | | | 7.00 | | 9.00 |
| Titanium Dioxide - microfine | 1.00 | | 3.00 | | 3.50 |
| Zinc Oxide - microfine | | | | 0.25 | |
| C₁₂₋₁₅ Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Biosaccaride Gum-1 | | 2.50 | 0.50 | | 2.00 |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume Oil | 0.20 | | 0.05 | 0.40 | |
| Demineralized Water | ad 100 | ad 100 | ad 100 | ad 100 | ad 100 |

WO Sunscreen emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Peroxide decomposer | 5.0 | 1.0 | 10.0 | 0.5 | 0.1 |
| Uvinul ® A Plus ™ | 2.00 | 1.50 | 0.75 | 1.00 | 2.10 |
| UvaSorb ® k2A | | 2.00 | | | |
| Ethylhexyl Bis- | | | | | |
| Isopentylbenzoxazolylphenyl Melamine | | | | | |
| Ethylhexyl Methoxycinnamate Uvinul ® | | | | | 5.00 |
| MC 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethylhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic | | 1.50 | | 1.00 | 1.00 |
| Acid | | | | | |
| Diethylhexyl 2,6-Naphthalate | | | 7.00 | | 4.00 |
| Titanium Dioxide - microfine | 1.00 | | 3.00 | | 3.50 |
| Zinc Oxide - microfine | | | | 0.25 | |
| Mineral Oil | | 12.00 | 10.00 | | 8.00 |
| C₁₂₋₁₅ Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate MgSO₄ | 1.00 | 0.50 | | 0.50 | |
| Magnesium Chloride MgCl₂ | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 1.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume Oil | 0.20 | | 0.40 | 0.35 | |
| Demineralized Water | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C₁₆₋₄₀ Alkyl Stearate | | 1.50 | 1.50 | 1.50 |
| Peroxide decomposer | 0.5 | 3.0 | 1.0 | 5.0 |
| Uvinul ® A Plus ™ | 2.00 | 1.50 | 0.75 | 9.00 |
| UvaSorb ® k2A | | 2.00 | | 4.00 |
| Uvinul ® Ethylhexyl Bis- | | | | |
| Isopentylbenzoxazolylphenyl | | | | |
| Melamine | | | | |
| Ethylhexyl Methoxycinnamate | | 3.00 | | |
| MC 80 | | | | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethylhexyl Triazone | 4.00 | | 3.00 | 4.00 |
| Uvinul ® T 150 | | | | |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | | | 7.00 | |
| Titanium Dioxide - microfine | 1.00 | | 3.00 | |
| Zinc Oxide - microfine | | | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume Oil, BHT | 0.10 | 0.25 | | 0.35 |
| Ricinus Communis (Castor) Oil | ad 100 | ad 100 | ad 100 | ad 100 |

PIT Emulsion

| | 1 | 2 | 3 | 4 | 5 | 6 | 7 | 8 |
|---|---|---|---|---|---|---|---|---|
| Glyceryl Monostearate | 0.50 | 2.00 | 3.00 | 5.00 | | 0.50 | 4.00 | |
| SE | | | | | | | | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2 Dioleate | | | | 0.75 | 0.30 | | | |
| Peroxide decomposer | 0.1 | 5.0 | 0.01 | 0.5 | 3.0 | 0.25 | 10.0 | 3.0 |
| Uvinul ® A Plus ™ | 2.00 | 1.50 | 0.75 | 1.00 | 2.10 | 4.50 | 5.00 | 2.10 |
| UvaSorb ® k2A | | | 4.00 | | | | 1.50 | |
| Ethylhexyl Bis- | | | | | | | | |
| Isopentylbenzoxazolyl | | | | | | | | |
| phenyl Melamine | | | | | | | | |
| Ethylhexyl | | | | | 5.00 | 6.00 | 8.00 | 5.00 |
| Methoxycinnamate | | | | | | | | |
| Uvinul ® MC 80 | | | | | | | | |
| Bis- | | 1.50 | | 2.00 | 2.50 | | 2.50 | 2.50 |
| Ethylhexyloxyphenol | | | | | | | | |
| Methoxyphenyl Triazine | | | | | | | | |
| Butyl | | | 2.00 | | 2.00 | 1.50 | | 2.00 |
| Methoxydibenzoyl- | | | | | | | | |
| methane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethylhexyl Triazone | 4.00 | | 3.00 | 4.00 | | 2.00 | | |
| Uvinul ® T 150 | | | | | | | | |
| Octocrylene | | 4.00 | | | | | 7.50 | |
| Diethylhexyl Butamido | 1.00 | | | 2.00 | 1.00 | | 1.00 | 1.00 |
| Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole | | | 0.50 | | | 1.00 | | |
| Trisiloxane | | | | | | | | |
| Terephthalylidene | | 1.50 | | 1.00 | 1.00 | | 0.50 | 1.00 |
| Dicamphor Sulfonic | | | | | | | | |
| Acid | | | | | | | | |
| Diethylhexyl 2,6- | | | 7.00 | | 10.00 | 7.50 | | 8.00 |
| Naphthalate | | | | | | | | |
| Titanium Dioxide - | 1.00 | | 3.00 | | 3.50 | | 1.50 | 3.50 |
| microfine | | | | | | | | |
| Zinc Oxide - microfine | | | | 0.25 | | 2.00 | | |
| C₁₂₋₁₅ Alkyl Benzoate | 3.50 | | | 6.35 | | | | 0.10 |
| Cocoglyceride | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C₁₋₅ Alkyl | | 0.50 | | | 2.00 | | | |
| Galactomannan | | | | | | | | |
| Hydrogenated Coco- | | | | | 3.00 | 4.00 | | |
| Glycerides | | | | | | | | |
| Behenoxy Dimethicone | | | | | | 1.50 | 2.00 | |
| VP/Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii | | 2.00 | | 3.60 | | 2.00 | | |
| (Shea Butter) | | | | | | | | |
| Iodopropyl | 0.12 | | | | 0.20 | | | |
| Butylcarbamate | | | | | | | | |
| Biosaccaride Gum-1 | | | | 0.10 | | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume Oil | 0.20 | | 0.20 | | 0.24 | 0.16 | 0.10 | 0.10 |
| Demineralized Water | ad | ad | ad | ad | ad | ad 100 | ad | ad |
| | 100 | 100 | 100 | 100 | 100 | | 100 | 100 |

Gel cream

| | 1 | 2 | 3 | 4 |
|---|---|---|---|---|
| Acrylates/C₁₀₋₃₀ Alkyl Acrylate | 0.40 | 0.35 | 0.40 | 0.35 |
| Crosspolymer | | | | |
| Carbomer | 0.20 | 0.22 | 0.20 | 0.22 |
| Luvigel ® EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| C₁₂₋₁₅ Alkylbenzoate | 4.00 | 4.50 | 4.00 | 4.50 |
| Caprylic/Capric Triglyceride | 3.00 | 3.50 | 3.00 | 3.50 |
| Titanium Dioxide - microfine | 1.00 | | 1.50 | |
| Zinc Oxide - microfine | | 2.00 | | 0.25 |
| Peroxide decomposer | 0.5 | 10.0 | 3.0 | 5.0 |
| Dihydroxyacetone | | | 3.00 | 5.00 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume Oil | 0.20 | | 0.20 | |
| Demineralized Water | ad 100 | ad 100 | ad 100 | ad 100 |

Adjust pH to 6.0

OW Self-tanning
formulations

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Peroxide decomposer | 0.1 | 0.5 | 0.025 | 5.0 | 3.0 | 10.0 | 1.0 |
| Dihydroxyacetone | | | 3.00 | 5.00 | | 4 | |
| Titanium Dioxide - | 1.00 | | | | 1.50 | | 1.50 |
| microfine | | | | | | | |
| Zinc Oxide - microfine | | | | 0.25 | | 2.00 | |
| C₁₂₋₁₅ Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii | | 2.00 | | | | | |
| (Shea) Butter | | | | | | | |
| VP/Hexadecene | 0.20 | | | 0.50 | | 1.00 | |
| Copolymer | | | | | | | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl | | | | 0.18 | 0.20 | | |
| Butylcarbamate | | | | | | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume Oil | 0.10 | 0.25 | 0.30 | | 0.40 | 0.20 | |
| Demineralized Water | ad 100 | ad 100 | ad 100 | ad 100 | ad 100 | ad100 | ad 100 |

OW Makeup

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Peroxide decomposer | 3.0 | 5.0 | 2.0 | 0.5 | 1.0 | 5.0 | 10.0 |
| Titanium Dioxide | 10.00 | 12.00 | 9.00 | 8.50 | 11.00 | 9.50 | 10.00 |
| Iron Oxide | 2.00 | 4.00 | 3.00 | 5.00 | 3.40 | 6.00 | 4.40 |
| Zinc Oxide | | 4.00 | | 2.00 | | 3.00 | |
| C₁₂₋₁₅ Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii | | 2.00 | | | | | |
| (Shea Butter) | | | | | | | |
| VP/Hexadecene | 0.20 | | | 0.50 | | 1.00 | |
| Copolymer | | | | | | | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soja (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl | | | | 0.18 | 0.20 | | |
| Butylcarbamate | | | | | | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume Oil | 0.10 | 0.25 | 0.30 | | 0.40 | 0.20 | |
| Demineralized Water | ad 100 | ad 100 | ad 100 | ad 100 | ad 100 | ad 100 | ad 100 |

Self-tanning hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C₁₀₋₃₀ Alkyl Acrylate | 0.50 | | 0.40 | 0.10 | 0.50 |
| Crosspolymer | | | | | |
| Xanthan Gum | | 0.30 | 0.15 | | |
| Peroxide decomposer | 3.0 | 1.0 | 0.5 | 0.1 | 5.0 |
| Dihydroxyacetone | | | 3.00 | 5.00 | |
| Uvinul ® A Plus ™ | 2.00 | 1.50 | 0.75 | 1.00 | 2.10 |
| Titanium Dioxide - microfine | 1.00 | | 1.00 | | 1.00 |
| Zinc Oxide - microfine | | 1.90 | | 0.25 | |
| C₁₂₋₁₅ Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume Oil | 0.20 | | 0.05 | 0.40 | |
| Demineralized Water | ad 100 | ad 100 | ad 100 | ad 100 | ad 100 |

Aftersun hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Luvigel ® EM | | 2.00 | | 2.50 | 2.00 |
| Acrylates/C₁₀₋₃₀ Alkyl Acrylate | 0.50 | 0.30 | 0.40 | 0.10 | 0.50 |
| Crosspolymer | | | | | |
| Xanthan Gum | | 0.30 | 0.15 | | |
| Peroxide decomposer | 0.1 | 5.0 | 0.5 | 3.0 | 1.0 |
| C₁₂₋₁₅ Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soja (Soybean) Oil | | | 1.50 | | 1.00 |
| Vitamin E Acetate | 0.50 | | 0.25 | | 1.00 |
| Glucosylrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume Oil | 0.20 | | 0.05 | 0.40 | |
| Demineralized Water | ad 100 | ad 100 | ad 100 | ad 100 | ad 100 |

WO Emulsions

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Peroxide decomposer | 5.0 | 10.0 | 0.1 | 0.5 | 1.0 |
| Uvinul ® A Plus ™ | 2.00 | 1.50 | 0.75 | 1.00 | 2.10 |
| Titanium Dioxide - microfine | 1.00 | | 3.00 | | 3.50 |
| Zinc Oxide - microfine | | 0.90 | | 0.25 | |
| Mineral Oil | | 12.00 | 10.00 | | 8.00 |
| C₁₂₋₁₅ Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soja (Soybean) Oil | | 1.00 | 1.50 | | 1.00 |
| Magnesium Sulfate | 1.00 | 0.50 | | 0.50 | |
| Magnesium Chloride | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 7.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume Oil | 0.20 | | 0.40 | 0.35 | |
| Demineralized Water | ad 100 | ad 100 | ad 100 | ad 100 | ad 100 |

Solids-stabilized emulsion
(Pickering Emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric Triglyceride | 9.00 | 9.00 | 6.00 | | |
| C₁₂₋₁₅ Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hectorite | 1.00 | 0.75 | | 0.50 | 0.25 |
| Cera Microcristallina + Paraffinum | | 0.35 | | | 5.00 |
| Liquidum | | | | | |
| Hydroxypropyl Methylcellulose | | | 0.10 | | 0.05 |
| Dimethicone | | | | | 3.00 |
| Peroxide decomposer | 1.0 | 0.5 | 0.1 | 3.0 | 5.0 |
| Titanium Dioxide + Alumina + Simethicone + Aqua | | 3.00 | | | |
| Titanium Dioxide + Trimethoxycaprylylsilane | | 2.00 | 4.00 | 2.00 | 4.00 |
| Silica Dimethyl Silylate | 2.50 | | | 6.00 | 2.50 |
| Boron Nitride | | | 1.00 | | |
| Starch/Sodium Metaphosphate Polymer | 2.00 | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyethanol | | | 0.20 | | |
| Hexamidine Diisethionate | | | 0.40 | 0.50 | 0.40 |
| Diazolidinyl Urea | | | | | 0.08 |
| Ethanol | | | 0.23 | 0.20 | |
| Perfume Oil | 5.00 | | 3.00 | 4.00 | |
| Demineralized Water | 0.20 | | 0.30 | 0.10 | |
| | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C₁₆₋₄₀ Alkyl Stearate | | 1.50 | 1.50 | 1.50 |
| Peroxide decomposer | 10.0 | 1.0 | 3.0 | 0.1 |
| Uvinul ® A Plus ™ | 2.00 | 1.50 | 0.75 | 9.00 |
| Titanium Dioxide - microfine | 1.00 | | 3.00 | |
| Zinc Oxide - microfine | | 1.00 | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume Oil, BHT | 0.10 | 0.25 | | 0.35 |
| Ricinus Communis (Castor) Oil | ad 100 | ad 100 | ad 100 | ad 100 |

Self-tanning PIT emulsions

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 | | | | 0.75 | 0.30 | | | |
| Peroxide decomposer | 0.1 | 0.5 | 0.01 | 5.0 | 0.5 | 3.0 | 0.025 | 10.0 |
| Dihydroxyacetone | | | 3.00 | 5.00 | | | 4.00 | |
| Uvinul ® A Plus ™ | 2.00 | 1.50 | 0.75 | 1.00 | 2.10 | 4.50 | 5.00 | 2.10 |
| Titanium Dioxide - microfine | 1.00 | | 1.50 | | 3.50 | | 1.50 | 1.00 |
| Zinc Oxide - microfine | | 1.00 | | 0.25 | | 2.00 | | 1.50 |
| C₁₂₋₁₅ Alkyl Benzoate | 3.50 | | | 6.35 | | | | 0.10 |
| Cocoglycerides | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C₁₋₅ Alkyl Galactomannan | | 0.50 | | | 2.00 | | | |
| Hydrogenated Coco- | | | | | 3.00 | 4.00 | | |
| Glycerides | | | | | | | | |
| Behenoxy Dimethicone | | | | | | 1.50 | 2.00 | |
| VP/Hexadecene Copolymer | | | | 1.00 | 1.20 | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii (Shea | | 2.00 | | 3.60 | | 2.00 | | |
| Butter) | | | | | | | | |
| Iodopropyl Butylcarbamate | 0.12 | | | | 0.20 | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume Oil | 0.20 | | 0.20 | | 0.24 | 0.16 | 0.10 | 0.10 |
| Demineralized Water | ad | ad | ad | ad | ad | ad 100 | ad | ad |
| | 100 | 100 | 100 | 100 | 100 | | 100 | 100 |

Oil gel

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butytene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Myristyl Myristate | 3.50 | 3.00 | 4.00 | 3.00 |
| Quaternium-8 Bentonite | 5.00 | 5.00 | 6.00 | 6.00 |
| Propylene Carbonate | 15.00 | 20.00 | 18.00 | 19.50 |
| Peroxide decomposer | 1.0 | 0.5 | 3.0 | 5.0 |
| Vitamin E Acetate | 0.50 | 1.00 | | |
| Ascorbyl Palmitate | 0.05 | | 0.05 | |
| Buxus Chinensis (Jojoba) Oil | 2.00 | 1.00 | | 1.00 |
| Perfume Oil, BHT | 0.10 | 0.25 | | 0.35 |
| Ricinus Communis (Castor) Oil | ad 100 | ad 100 | ad 100 | ad 100 |

### Example 23

In the following formulations, cosmetic sunscreen preparations comprising a combination of at least one inorganic pigment, preferably zinc oxide and/or titanium dioxide, and organic UV-A and UV-B filters are described.

The formulations given below are prepared in a customary manner known to the person skilled in the art.

The content of peroxide decomposer according to compound I.1-I.3, for example compounds specified in table 1, refers to 100% of active ingredient. The active ingredients according to the invention can be used either in pure form or as aqueous solution. In the case of the aqueous solution, the content of water dem. in the particular formulation has to be adjusted.

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 4.10 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 1.00 | Sepigel 305 | Polyacrylamide, C₁₃₋₁₄ Isoparaffin, |
| | | | Laureth-7 |

1% Peroxide decomposer

| | |
|---|---|
| q.s. | Preservative |

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, Polysorbate 60 |

1% Peroxide decomposer

| | |
|---|---|
| q.s. | Preservative |

| | | | |
|---|---|---|---|
| A | 7.50 | Uvinul MC 80 | Ethylhexyl Cinnamate |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, Polysorbate 60 |

1% Peroxide decomposer

| | |
|---|---|
| q.s. | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate; PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, Polysorbate 60 |

1% Peroxide decomposer

| | |
|---|---|
| q.s. | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, Polysorbate 60 |

1% Peroxide decomposer

| | |
|---|---|
| q.s. | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, Polysorbate 60 |

1% Peroxide decomposer

| | |
|---|---|
| q.s. | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 |
| | | | Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, |
| | | | Methicone/Dimethicone Copolymer |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.70 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, Polysorbate 60 |

0.5% Peroxide decomposer

| | |
|---|---|
| q.s. | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 67.10 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, |
| | | | Polysorbate 60 |

0.1% Peroxide decomposer

| | |
|---|---|
| q.s. | Preservative |

| | | | |
|---|---|---|---|
| A | 5.00 | Uvinul N 539 T | Octocrylene |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, Methicone/ |
| | | | Dimethicone Copolymer |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 2.00 | Simulgel NS | Hydroxyethyl Acrylate/Sodium |
| | | | Acryloyldimethyl Taurate |
| | | | Copolymer, Squalane, Polysorbate 60 |

1% Peroxide decomposer

| | |
|---|---|
| q.s. | Preservative |

| | | | |
|---|---|---|---|
| A | 5.00 | Uvinul N 539 T | Octocrylene |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 65.20 | Water dem. | Demineralized Water |
| D | 2.00 | Simulgel NS | Hydroxyethyl Acrylate/Sodium |
| | | | Acryloyldimethyl Taurate Copolymer, |
| | | | Squalane, Polysorbate 60 |

2% Peroxide decomposer

| | |
|---|---|
| q.s. | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.70 | Water dem. | Demineralized Water |
| D | 2.00 | Simulgel NS | Hydroxyethyl Acrylate/Sodium |
| | | | Acryloyldimethyl Taurate Copolymer, |
| | | | Squalane, Polysorbate 60 |

0.5% Peroxide decomposer

| | |
|---|---|
| q.s. | Preservative |

| | | | |
|---|---|---|---|
| A | 5.00 | Uvinul N 539 T | Octocrylene |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 2.0 | Mexoryl SX | Terephthalidene Dicamphor Sulfonic |
| | | | Acid |
| | 66.20 | Water dem. | Demineralized Water |
| D | 2.00 | Simulgel NS | Hydroxyethyl Acrylate/Sodium |
| | | | Acryloyldimethyl Taurate Copolymer, |
| | | | Squalane, Polysorbate 60 |

1% Peroxide decomposer

| | |
|---|---|
| q.s. | Preservative |

| | | | |
|---|---|---|---|
| A | 5.00 | Uvinul N 539 T | Octocrylene |
| | 2.00 | Uvinul A Plus | Diethylamino Hydrobenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and)Diphenyl Capryl |
| | | | Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 2.0 | Mexoryl SX | Terephthalidene Dicamphor |
| | | | Sulfonic Acid |
| | 65.20 | Water dem. | Demineralized Water |
| D | 2.00 | Simulgel NS | Hydroxyethyl Acrylate/Sodium |
| | | | Acryloyldimethyl Taurate Copolymer, |
| | | | Squalane, Polysorbate 60 |

2% Peroxide decomposer

| | |
|---|---|
| q.s. | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 2.00 | Mexoryl XL | Drometrizole Trisiloxane |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 67.10 | Water dem. | Demineralized Water |
| D | 2.00 | Simulgel NS | Hydroxyethyl Acrylate/Sodium |
| | | | Acryloyldimethyl Taurate Copolymer, |
| | | | Squalane, Polysorbate 60 |

0.1% Peroxide decomposer

| | |
|---|---|
| q.s. | Preservative |

| | | | |
|---|---|---|---|
| A | 5.00 | Uvinul N 539 T | Octocrylene |
| | 2.00 | Tinosorb S | Bis-Ethylhexyloxyphenol |
| | | | Methoxyphenyl Triazine |
| | 0.80 | Rylo PG 11 | Polyglyceryl Dimer Soyate |
| | 1.00 | Span 60 | Sorbitan Stearate |
| | 0.50 | Vitamin E Acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C₁₂₋₁₅ Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water dem. | Demineralized Water |
| D | 2.00 | Simulgel NS | Hydroxyethyl Acrylate/Sodium |
| | | | Acryloyldimethyl Taurate Copolymer, |
| | | | Squalane, Polysorbate 60 |

1% Peroxide decomposer

| | |
|---|---|
| q.s. | Preservative |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl |
| | | | Methicone |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water dem. | Demineralized Water |
| | q.s. | Preservative | |
| | 0.50 | Vitamin E Acetate | Tocopheryl Acetate |

2% Peroxide decomposer

| | | |
|---|---|---|
| 1.00 | RetiSTAR | Caprylic/Capric Triglyceride, Sodium |
| | | Ascorbate, Tocopherol, Retinol |

| | | | |
|---|---|---|---|
| A | 7.50 | Uvinul MC 80 | Ethylhexyl Methoxycinnamate |
| | 2.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica Hydrate, |
| | | | Alumina Hydrate, |
| | | | Methicone/Dimethicone Copolymer |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water dem. | Demineralized Water |
| | q.s. | Preservative | |
| | 0.50 | Vitamin E Acetate | Tocopheryl Acetate |

1% Peroxide decomposer

| | | |
|---|---|---|
| 2.00 | RetiSTAR | Caprylic/Capric Triglyceride, Sodium |
| | | Ascorbate, Tocopherol, Retinol |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene | Propylene Glycol |
| | | Glycol Care | |
| | 1.00 | D-Panthenol 50 P | Panthenol, Propylene Glycol |
| | 0.20 | Keltrol | Xanthan Gum |
| | 0.50 | Simulgel 600 | Acrylamide/Sodium |
| | | | Acryloyldimethyltaurate |
| | | | Copolymer, Isohexadecane, |
| | | | Polysorbate 80 |
| | ad 100 | Water dem. | Demineralized Water |
| | q.s. | Preservative | |
| | 0.50 | Vitamin E Acetate | Tocopheryl Acetate |

1% Peroxide decomposer

| | | |
|---|---|---|
| 1.00 | RetiSTAR | Caprylic/Capric Triglyceride, Sodium |
| | | Ascorbate, Tocopherol, Retinol |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl Hexyl Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene Glycol Care | Propylene Glycol |
| | 0.20 | Keltrol | Xanthan Gum |
| | 0.50 | Simulgel 600 | Acrylamide/Sodium Acryloyldimethyltaurate |
| | | | Copolymer, Isohexadecane, Polysorbate 80 |
| | ad 100 | Water dem. | Demineralized Water |
| | q.s. | Preservative | |
| | 0.50 | Vitamin E Acetate | Tocopheryl Acetate |

2% Peroxide decomposer

| | | |
|---|---|---|
| 1.00 | RetiSTAR | Caprylic/Capric Triglyceride, Sodium |
| | | Ascorbate, Tocopherol, Retinol |

| | | | |
|---|---|---|---|
| A | 1.00 | Abil Care 85 | Bis-PEG/PPG-16/16 PEG/PPG-16/16 |
| | | | Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning | Cyclopentasiloxane, Cyclohexasiloxane |
| | | 345 Fluid | |
| | 5.00 | Uvinul N 539 T | Octocrylene |
| B | 3.00 | Z-COTE MAX | Zinc Oxide (and) Diphenyl Capryl |
| | | | Methicone |
| C | 5.00 | 1,2-Propylene | Propylene Glycol |
| | | Glycol | |
| | 1.00 | Mexoryl SX | Terephthalidene Dicamphor Sulfanic |
| | | | Acid |
| | 0.20 | Keltrol | Xanthan Gum |
| | 0.06 | Edeta BD | Disodium EDTA |
| | 0.04 | TEA | Triethanolamine |
| | 0.30 | Carbopol Ultrez 10P | Carbomer |
| | 63.50 | Water dem. | Demineralized Water |

1% Peroxide decomposer

| | | | |
|---|---|---|---|
| D | q.s. | Perfume Oil | |
| | 0.50 | Glydant | DMDM Hydantoin |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica Hydrate, |
| | | | Alumina Hydrate, |
| | | | Methicone/Dimethicone Copolymer |
| C | 4.00 | 1,2-Propylene | Propylene Glycol |
| | | Glycol Care | |
| | 0.20 | Keltrol | Xanthan Gum |
| | 2.00 | Simulgel NS | Hydroxyethyl Acrylate/Sodium |
| | | | Acryloyldimethyl Taurate Copolymer, |
| | | | Squalane, Polysorbate 60 |
| | 66.30 | Water dem. | Demineralized Water |
| | q.s. | Preservative | |
| | 0.50 | Vitamin E Acetate | Tocopheryl Acetate |

0.5% Peroxide decomposer

| | | |
|---|---|---|
| 1.00 | RetiSTAR | Caprylic/Capric Triglyceride, Sodium |
| | | Ascorbate, Tocopherol, Retinol |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | Z-COTE MAX | Zinc Oxide (and) Diphenyl Capryl |
| | | | Methicone |
| C | 0.20 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 65.10 | Water dem. | Demineralized Water |
| | 0.20 | Edeta BD | Disodium EDTA |
| | 5.00 | 1,2-Propylene | Propylene Glycol |
| | | Glycol Care | |

0.5% Peroxide decomposer

| | | | |
|---|---|---|---|
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 65.10 | Water dem. | Demineralized Water |
| | 0.20 | Edeta BD | Disodium EDTA |
| | 5.00 | 1,2-Propylene | Propylene Glycol |
| | | Glycol Care | |

0.5% Peroxide decomposer

| | | | |
|---|---|---|---|
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica |
| | | | Hydrate, Alumina Hydrate, |
| | | | Methicone/Dimethicone |
| | | | Copolymer |
| C | 0.50 | Keltrol | Xanthan Gum |
| | 0.50 | Simulgel NS | Hydroxyethyl Acrylate/Sodium |
| | | | Acryloyldimethyl Taurate |
| | | | Copolymer, Squalane, |
| | | | Polysorbate 60 |
| | 64.60 | Water dem. | Demineralized Water |
| | 0.20 | Edeta BD | Disodium EDTA |
| | 5.00 | 1,2-Propylene Glycol Care | Propylene Glycol |

1% Peroxide decomposer

| | | | |
|---|---|---|---|
| D | 1.00 | Vitamin E Acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica Hydrate, |
| | | | Alumina Hydrate, |
| | | | Methicone/Dimethicone |
| | | | Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 64.10 | Water dem. | Demineralized Water |
| | 2.00 | Neoheliopan AP | Disodium Phenyl Dibenzimidazole |
| | | | Tetrasulfonate |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |

1% Peroxide decomposer

| | |
|---|---|
| q.s. | Preservative |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 7.00 | Uvinul N 539 T | Octocrylene |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica Hydrate, |
| | | | Alumina Hydrate, |
| | | | Methicone/Dimethicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 64.60 | Water dem. | Demineralized Water |
| | 1.00 | Mexoryl SX | Terephthalidene Dicamphor Sulfonic |
| | | | Acid |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |

1% Peroxide decomposer

| | |
|---|---|
| q.s. | Preservative |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 2.00 | Mexoryl XL | Drometrizole Trisiloxane |
| | 5.00 | Uvinul N 539 T | Octocrylene |
| | 1.00 | Ganex V-220 | VP/Eicosene Copolymer |
| | 7.00 | Isopropyl Palmitate | Isopropyl Palmitate |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 65.10 | Water dem. | Demineralized Water |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |

0.5% Proxide decomposer

| | |
|---|---|
| q.s. | Preservative |

| | | | |
|---|---|---|---|
| | 1.00 | Urea | Urea |
| | 2.00 | Neoheliopan AP | Disodium Phenyl Dibenzimidazole |
| | | | Tetrasulfonate |
| | 2.00 | Glycerin 87% | Glycerin |
| | 1.20 | Aristoflex AVC | Ammonium |
| | | | Acryloyldimethyltaurate/VP |
| | | | Copolymer |
| | 71.30 | Water dem. | Demineralized Water |
| B | 3.00 | Cremophor CO 40 | PEG-40 Hydrogenated Castor Oil |
| | q.s. | Perfume Oil | |
| | 5.00 | Miglyol 812 | Caprylic/Capric Triglyceride |
| | 2.00 | Uvinul N 539 T | Octocrylene |
| | 8.00 | Z-COTE MAX | Zinc Oxide (and) Diphenyl Capryl |
| | | | Methicone |

5% Peroxide decomposer

| | |
|---|---|
| q.s. | Preservative |

| | | |
|---|---|---|
| 5.00 | Z-COTE MAX | Zinc Oxide (and) Diphenyl Capryl |
| | | Methicone |
| 2.00 | Parsol 1789 | Butyl Methoxydibenzoylmethane |
| 1.00 | Uvinul T150 | Ethylhexyl Triazone |
| 5.00 | Uvinul MC 80 | Ethylhexyl Methoxycinnamate |
| 2.00 | Uvinul T 539 N | Octocrylene |
| 0.50 | Abil 350 | Dimethicone |
| 2.75 | Carnicowachs LT 20 | Carnauba (Copernica Cerifera) Wax, |
| | | Paraffin |
| 3.70 | Candelillawachs LT 281 LJ | Candelilla (Euphorbia Cerifera) Wax |
| 1.80 | Bienenwachs 3050 PH | Bees Wax |
| 3.20 | TeCero-Wachs 30445 | Microcrystalline Wax |
| 3.20 | TeCero-Wachs 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E Acetate | Tocopheryl Acetate |
| 0.42 | D,L-Alpha-Tocopherol | Tocopherol |

1% Peroxide decomposer

| | | |
|---|---|---|
| 40.38 | Ricinus Oil | Castor (Ricinus Communis) Oil |

| | | |
|---|---|---|
| 5.00 | T-Lite SF | Titanium Dioxide, Alumina Hydrate, |
| | | Dimethicone/Methicone Copolymer |
| 2.00 | Parsol 1789 | Butyl Methoxydibenzoylmethane |
| 1.00 | Uvinul T150 | Ethylhexyl Triazone |
| 3.00 | Uvinul MC 80 | Ethylhexyl Methoxycinnamate |
| 2.00 | Uvinul N 539 T | Octocrylene |
| 0.50 | Abil 350 | Dimethicone |
| 2.75 | Carnicowachs LT 20 | Carnauba (Copernica Cerifera) Wax, |
| | | Paraffin |
| 3.70 | Candelillawachs LT 281 LJ | Candelilla (Euphorbia Cerifera) Wax |
| 1.80 | Bienenwachs 3050 PH | Bees Wax |
| 3.20 | TeCero-Wachs 30445 | Microcrystalline Wax |
| 3.20 | TeCero-Wachs 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E Acetate | Tocopheryl Acetate |
| 0.42 | D,L-Alpha-Tocopherol | Tocopherol |

1% Peroxide decomposer

| | | |
|---|---|---|
| 40.38 | Ricinus Oil | Castor (Ricinus Communis) Oil |

| | | |
|---|---|---|
| 5.00 | T-Lite SF-S | Titanium Dioxide, Silica Hydrate, |
| | | Alumina Hydrate, |
| | | Methicone/Dimethicone Copolymer |
| 2.00 | Uvinul A Plus | Diethylamino Hydroxybenzoyl Hexyl |
| | | Benzoate |
| 2.00 | Mexoryl XL | Drometrizole Trisiloxane |
| 3.00 | Uvinul MC 80 | Ethylhexyl Methoxycinnamate |
| 0.50 | Abil 350 | Dimethicone |
| 2.75 | Carnicowachs LT 20 | Carnauba (Copernica Cerifera) |
| | | Wax, Paraffin |
| 3.70 | Candelillawachs LT 281 LJ | Candelilla (Euphorbia Cerifera) |
| | | Wax |
| 1.80 | Bienenwachs 3050 PH | Bees Wax |
| 3.20 | TeCero-Wachs 30445 | Microcrystalline Wax |
| 3.20 | TeCero-Wachs 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E Acetate | Tocopheryl Acetate |
| 0.42 | D,L-Alpha-Tocopherol | Tocopherol |

2% Peroxide decomposer

| | | |
|---|---|---|
| 39.38 | Ricinus Oil | Castor (Ricinus Communis) Oil |

| | | | |
|---|---|---|---|
| A | 6.00 | Cremophor WO 7 | PEG-7 Hydrogenated Castor Oil |
| | 2.00 | Elfacos ST 9 | PEG-45/Dodecyl Glycol Copolymer |
| | 3.00 | Isopropyl Myristate | Isopropyl Myristate |
| | 8.00 | Jojoba Oil | Simmondsia Chinensis (Jojoba) Seed |
| | | | Oil |
| | 4.00 | Uvinul MC 80 | Ethylhexyl Methoxycinnamate |
| | 2.00 | Uvinul A Plus | Diethylamino Hydroxybenzoyl Hexyl |
| | | | Benzoate |
| | 1.00 | Abil 350 | Dimethicone |
| B | 5.00 | Z-COTE MAX | Zinc Oxide (and) Diphenyl Capryl |
| | | | Methicone |
| | 3.00 | T-Lite SF | Titanium Dioxide, Alumina Hydrate, |
| | | | Dimethicone/Methicone Copolymer |
| C | 0.20 | Edeta BD | Disodium EDTA |
| | 5.00 | Glycerin 87% | Glycerin |
| | 0.30 | Chemag 2000 | Imidazolidinyl Urea |
| | 59.00 | Water dem. | Demineralized Water |
| D | q.s. | Perfume Oil | |

1% Peroxide decomposer

| | |
|---|---|
| q.s. | Preservative |

| | | | |
|---|---|---|---|
| | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Uvinul T 150 | Ethylhexyl Triazone |
| | 1.00 | Ganex V-220 | VP/Eicosene Copolymer |
| | 7.00 | Isopropyl Myristate | Isopropyl Myristate |
| B | 5.00 | Z-COTE MAX | Zinc Oxide (and) Diphenyl Capryl |
| | | | Methicone |
| C | 0.20 | Keltrol | Xanthan Gum |
| | 0.50 | Simulgel NS | Hydroxyethyl Acrylate/Sodium |
| | | | Acryloyldimethyl Taurate |
| | | | Copolymer, Squalane, |
| | | | Polysorbate 60 |
| | 61.10 | Water dem. | Demineralized Water |
| | 0.20 | Edeta BD | Disodium EDTA |
| | 5.00 | 1,2-Propylene | Propylene Glycol |
| | | Glycol Care | |
| D | 1.00 | Vitamin E Acetate | Tocopheryl Acetate |

1.5% Peroxide decomposer

| | |
|---|---|
| q.s. | Preservative |

| | | | |
|---|---|---|---|
| A | 2.00 | Luvitol EHO | Cetearyl Ethylhexanoate |
| | 5.00 | Paraffin Oil | Mineral Oil |
| | 5.00 | Z-COTE MAX | Zinc Oxide (and) Diphenyl |
| | | | Capryl Methicone |
| B | 1.00 | Cremophor A 6 | Ceteareth-6, Stearyl Alcohol |
| | 2.00 | Cremophor A 25 | Ceteareth-25 |
| | 3.00 | Glycerin monostearate | Glyceryl Stearate |
| | 4.00 | Cetylstearyl alcohol | Cetearyl Alcohol |
| C | 5.00 | 1,2-Propylene Glycol Care | Propylene Glycol |
| | 7.50 | Luviquat Care | Polyquaternium-44 |
| | q.s. | Preservative | |
| | q.s. | Perfume Oil | |

1% Peroxide decomposer

| | | |
|---|---|---|
| 64.50 | Water dem. | Demineralized Water |

| | | |
|---|---|---|
| 5.00 | T-Lite SF | Titanium Dioxide, Alumina |
| | | Hydrate, Dimethicone/Methicone |
| | | Copolymer |
| 10.00 | Z-COTE MAX | Zinc Oxide (and) Diphenyl Capryl |
| | | Methicone |
| 12.00 | Bienenwachs 3044 PH | Bees Wax |
| 8.00 | Candelillawachs LT 281 LJ | Candelilla (Euphorbia Cerifera) |
| | | Wax |
| 5.00 | Tegin | Glyceryl Stearate SE |
| 5.00 | Dow Corning 345 Fluid | Cyclopentasiloxane, |
| | | Cyclohexasiloxane |
| 5.00 | Witconol APM | PPG-3 Myristyl Ether |
| 5.00 | Softisan 154 | Hydrogenated Palm Oil |
| 8.00 | Paraffin Oil, high viscosity | Mineral Oil |
| 3.00 | Vaseline | Petrolatum |

2% Peroxide decomposer

| | | |
|---|---|---|
| 32.00 | Ricinus Oil | Castor (Ricinus Communis) Oil |

| | | |
|---|---|---|
| 3.00 | Uvinul MC 80 | Ethylhexyl Methoxycinnamate |
| 2.00 | Uvinul T 150 | Ethylhexyl Triazone |
| 2.00 | Uvinul A Plus | Diethylamino Hydroxybenzoyl |
| | | Hexyl Benzoate |
| 10.00 | Z-COTE MAX | Zinc Oxide (and) Diphenyl Capryl |
| | | Methicone |
| 12.00 | Bienenwachs 3044 PH | Bees Wax |
| 3.00 | Vaseline | Petrolatum |
| 8.00 | Candelillawachs LT 281 LJ | Candelilla (Euphorbia Cerifera) |
| | | Wax |
| 8.00 | Paraffin Oil, high viscosity | Mineral Oil |
| 5.00 | Tegin | Glyceryl Stearate SE |
| 5.00 | Softisan 154 | Hydrogenated Palm Oil |
| 5.00 | Witconol APM | PPG-3 Myristyl Ether |
| 5.00 | Dow Corning 345 Fluid | Cyclopentasiloxane, |
| | | Cyclohexasiloxane |

0.1% Peroxide decomposer

| | | |
|---|---|---|
| 28.90 | Ricinus Oil | Castor (Ricinus Communis) Oil |

| | | | |
|---|---|---|---|
| | 5.00 | T-Lite SF | Titanium Dioxide, Alumina |
| | | | Hydrate, Dimethicone/Methicone |
| | | | Copolymer |
| | 6.00 | Finsolv TN | C12-15 Alkyl Benzoate |
| | 10.00 | Uvinul MC 80 | Ethylhexyl Methoxycinnamate |
| | 6.00 | Miglyol 812 | Caprylic/Capric Triglyceride |
| | 5.00 | Arlacel P 135 | PEG-30 Dipolyhydroxystearate |
| | 2.00 | Ganex V 216 | PVP/Hexadecene Copolymer |
| | 2.00 | Elfacos ST 9 | PEG-45/Dodecyl Glycol |
| | | | Copolymer |
| B | 3.00 | 1,2-Propylene | Propylene Glycol |
| | | Glycol Care | |
| | 0.10 | Edeta BD | Disodium EDTA |
| | 1.00 | Magnesium Sulfate | Magnesium Sulfate |
| | | 7-Hydrate | |
| | 59.40 | Water dem. | Demineralized Water |

0.5% Peroxide decomposer

| | |
|---|---|
| q.s. | Preservative |

| | | | |
|---|---|---|---|
| A | 4.00 | Dehymuls SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 |
| | | | Dipolyhydroxystearate |
| | 8.00 | Finsolv TN | C₁₂₋₁₅ Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | Sulfate 7-Hydrate | |
| | ad 100 | Water dem. | Demineralized Water |

1.5% Peroxide decomposer

| | | |
|---|---|---|
| D | q.s. | Preservative |

| | | | |
|---|---|---|---|
| A | 4.00 | Dehymuls SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 |
| | | | Dipolyhydroxystearate |
| | 8.00 | Finsolv TN | C₁₂₋₁₅ Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | T-Lite SF-S | Titanium Dioxide, Silica Hydrate, |
| | | | Alumina Hydrate, |
| | | | Methicone/Dimethicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | Sulfate 7-Hydrate | |

1.0% Peroxide decomposer

| | | | |
|---|---|---|---|
| | ad 100 | Water dem. | Demineralized Water |
| D | q.s. | Preservative | |

| | | | |
|---|---|---|---|
| A | 4.00 | Dehymuls SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 |
| | | | Dipolyhydroxystearate |
| | 8.00 | Finsolv TN | C₁₂₋₁₅ Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl |
| | | | Hexyl Benzoate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | Sulfate 7-Hydrate | |

0.1% Peroxide decomposer

| | | |
|---|---|---|
| ad 100 | Water dem. | Demineralized Water |
| q.s. | Preservative | |

| | | | |
|---|---|---|---|
| A | 4.00 | Dehymuls SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 |
| | | | Dipolyhydroxystearate |
| | 8.00 | Finsolv TN | C₁₂₋₁₅ Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl |
| | | | Hexyl Benzoate |
| | 2.00 | Uvinul N 539 T | Octocrylene |
| B | 5.00 | T-Lite SF | Titanium Dioxide, Alumina |
| | | | Hydrate, Dimethicone/Methicone |
| | | | Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | Sulfate 7-Hydrate | |
| | ad 100 | Water dem. | Demineralized Water |

0.2% Peroxide decomposer

| | |
|---|---|
| q.s. | Preservative |

| | | | |
|---|---|---|---|
| A | 4.00 | Dehymuls SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 |
| | | | Dipolyhydroxystearate |
| | 8.00 | Finsolv TN | C₁₂₋₁₅ Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl |
| | | | Hexyl Benzoate |
| | 2.00 | Uvinul N 539 T | Octocrylene |
| B | 5.00 | T-Lite SF-S | Titanium Dioxide, Silica Hydrate, |
| | | | Alumina Hydrate, |
| | | | Methicone/Dimethicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium Sulfate | Magnesium Sulfate |
| | | 7-Hydrate | |

0.5% Peroxide decomposer

| | | |
|---|---|---|
| ad 100 | Water dem. | Demineralized Water |

q.s. Preservative

| | | | |
|---|---|---|---|
| A | 4.00 | Dehymuls SBL | Polyglyceryl-2 |
| | | | Dipolyhydroxystearate, Dicaprylyl |
| | | | Ether, Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 |
| | | | Dipolyhydroxystearate |
| | 8.00 | Finsolv TN | C₁₂₋₁₅ Alkyl Benzoate |
| | 4.00 | Miglyol 812 | Caprylic/Capric Triglyceride |
| | 7.00 | Uvinul MC 80 | Ethylhexyl Methoxycinnamate |
| | 2.00 | Mexoryl XL | Drometrizole Trisiloxane |
| B | 5.00 | Z-COTE MAX | Titanium Dioxide, Alumina Hydrate, |
| | | | Dimethicone/Methicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | Sulfate 7-Hydrate | |
| | ad 100 | Water dem. | Demineralized Water |

0.01% Peroxide decomposer

| | |
|---|---|
| q.s. | Preservative |

| | | | |
|---|---|---|---|
| A | 7.50 | Uvinul MC 80 | Ethylhexyl Methoxycinnamate |
| | 3.00 | Uvinul N 539 T | Octocrylene |
| | 2.00 | Uvinul A Plus | Diethylamino Hydroxybenzoyl |
| | | | Hexyl Benzoate |
| | 1.00 | Cremophor CO 40 | PEG-40 Hydrogenated Castor Oil |
| | 10.00 | Miglyol 812 | Caprylic/Capric Triglyceride |
| | 1.50 | Dow Corning | Cyclopentasiloxane, |
| | | 345 Fluid | Cyclohexasiloxane |
| B | 3.50 | Luvigel EM | Caprylic/Capric Triglyceride, |
| | | | Sodium Acrylates Copolymer |
| C | 46.00 | Water dem. | Demineralized Water |
| D | 5.00 | 1,2-Propylene | Propylene Glycol |
| | | Glycol Care | |
| | 0.50 | Cremophor A 25 | Ceteareth-25 |

0.05% Peroxide decomposer

| | | |
|---|---|---|
| 20.00 | Ethanol 96% | Alcohol |

| | | | |
|---|---|---|---|
| A | 1.00 | Uvinul A Plus | Diethylamino Hydroxybenzoyl |
| | | | Hexyl Benzoate |
| | 1.00 | Tinosorb S | Bis-Ethylhexyloxyphenol |
| | | | Methoxyphenyl Triazine |
| | 3.00 | Uvinul MC 80 | Ethylhexyl Methoxycinnamate |
| | 8.00 | Miglyol 812 | Caprylic/Capric Triglyceride |
| | 1.50 | Dow Corning | Dimethicone |
| | | 350 Fluid | |
| | 3.00 | Z-COTE MAX | Titanium Dioxide, Alumina |
| | | | Hydrate, Dimethicone/Methicone |
| | | | Copolymer |
| | 3.00 | Finsolv TN | C₁₂₋₁₅ Alkyl Benzoate |
| | 1.00 | Cremophor CO 40 | PEG-40 Hydrogenated Castor Oil |
| B | 2.00 | Luvigel EM | Caprylic/Capric Triglyceride, |
| | | | Sodium Acrylates Copolymer |
| C | 54.80 | Water dem. | Demineralized Water |
| D | 15.00 | Ethanol 96% | Alcohol |
| | 5.00 | 1,2-Propylene | Propylene Glycol |
| | | Glycol Care | |
| | 0.50 | Cremophor A 25 | Ceteareth-25 |

1.0% Peroxide decomposer

| | | |
|---|---|---|
| 1.00 | Vitamin E Acetate | Tocopheryl Acetate |
| 0.20 | Bisabolol rac. | Bisabolol |

| | | | |
|---|---|---|---|
| A | 4.00 | Dehymuls SBL | Polyglyceryl-2 |
| | | | Dipolyhydroxystearate, |
| | | | Dicaprylyl Ether, |
| | | | Cocoglycerides, Sorbitan |
| | | | Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 |
| | | | Dipolyhydroxystearate |
| | 6.00 | Finsolv TN | C₁₂₋₁₅ Alkyl Benzoate |
| | 6.00 | Miglyol 812 | Caprylic/Capric Triglyceride |
| | 5.00 | Uvinul MC 80 | Ethylhexyl Methoxycinnamate |
| | 3.00 | Neoheliopan HMS | Homosalate |
| B | 5.00 | Z-COTE MAX | Zinc Oxide (and) Diphenyl |
| | | | Capryl Methicone |
| C | 3.00 | 1,2-Propylene | Propylene Glycol |
| | | Glycol Care | |
| | 0.30 | Chemag 2000 | Imidazolidinyl Urea |
| | 1.00 | Magnesium | Magnesium Sulfate |
| | | Sulfate 7-Hydrate | |
| | 65.10 | Water dem. | Demineralized Water |

0.1% Peroxide decomposer

| | |
|---|---|
| q.s. | Preservative |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| | 0.50 | Vitamin E Acetate | Tocopheryl Acetate |
| B | 5.00 | Isohexadecane | Isohexadecane |
| | 5.00 | Cosmacol EMI | Di-C₁₂₋₁₃ Alkyl Malate |
| | 3.50 | Cetiol SN | Cetearyl Isononanoate |
| | 1.00 | Ganex V-220 | VP/Eicosene Copolymer |
| | 5.00 | T-Lite SF | Titanium Dioxide, Alumina |
| | | | Hydrate, Dimethicone/Methicone |
| | | | Copolymer |
| C | 60.00 | Water dem. | Water |
| | 5.00 | Glycerin 87% | Glycerin |
| | 1.00 | Lanette E | Sodium Cetearyl Sulfate |
| | 0.30 | Keltrol | Xanthan Gum |

0.2% Peroxide decomposer

| | | |
|---|---|---|
| D | q.s. | Preservative |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C₁₂₋₁₃ Alkyl Malate |
| | 3.50 | Cetiol SN | Cetearyl Isononanoate |
| | 1.00 | Ganex V-220 | VP/Eicosene Copolymer |
| | 5.00 | T-Lite SF | Titanium Dioxide, Alumina |
| | | | Hydrate, |
| | | | Dimethicone/Methicone |
| | | | Copolymer |
| C | 59.20 | Water dem. | Demineralized Water |
| | 5.00 | Glycerin 87% | Glycerin |
| | 1.00 | Lanette E | Sodium Cetearyl Sulfate |
| | 0.30 | Keltrol | Xanthan Gum |

1.0% Peroxide decomposer

| | | |
|---|---|---|
| D | q.s. | Preservative |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Neoheliopan OS | Octisalate |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C₁₂₋₁₃ Alkyl Malate |
| | 3.50 | Cetiol SN | Cetearyl Isononanoate |
| | 1.00 | Ganex V-220 | VP/Eicosene Copolymer |
| | 5.00 | Z-COTE MAX | Zinc Oxide (and) Diphenyl |
| | | | Capryl Methicone |
| C | 60.15 | Water dem. | Aqua dem |
| | 5.00 | Glycerin 87% | Glycerin |
| | 1.00 | Lanette E | Sodium Cetearyl Sulfate |
| | 0.30 | Keltrol | Xanthan Gum |

0.05% Peroxide decomposer

| | | |
|---|---|---|
| D | q.s. | Preservative |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C₁₂₋₁₃ Alkyl Malate |
| | 3.50 | Cetiol SN | Cetearyl Isononanoate |
| | 1.00 | Ganex V-220 | VP/Eicosene Copolymer |
| | 5.00 | Z-COTE MAX | Zinc Oxide (and) Diphenyl |
| | | | Capryl Methicone |
| C | 60.19 | Water dem. | Demineralized Water |
| | 5.00 | Glycerin 87% | Glycerin |
| | 1.00 | Lanette E | Sodium Cetearyl Sulfate |
| | 0.30 | Keltrol | Xanthan Gum |

0.01% Peroxide decomposer

| | | |
|---|---|---|
| D | q.s. | Preservative |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E Acetate | Tocopheryl Acetate |
| B | 5.00 | Isohexadecane | Isohexadecane |
| | 5.00 | Cosmacol EMI | Di-C₁₂₋₁₃ Alkyl Malate |
| | 3.50 | Cetiol SN | Cetearyl Isononanoate |
| | 1.00 | Ganex V-220 | VP/Eicosene Copolymer |
| | 5.00 | T-Lite SF-S | Titanium Dioxide, Silica |
| | | | Hydrate, Alumina Hydrate, |
| | | | Methicone/Dimethicone |
| | | | Copolymer |
| C | 59.20 | Water dem. | Demineralized Water |
| | 5.00 | Glycerin 87% | Glycerin |
| | 1.00 | Lanette E | Sodium Cetearyl Sulfate |
| | 0.30 | Keltrol | Xanthan Gum |

1.0% Peroxide decomposer

| | | |
|---|---|---|
| D | q.s. | Preservative |

| | | | |
|---|---|---|---|
| A | 8.00 | Cetiol B | Dibutyl Adipate |
| | 8.00 | Finsolv TN | C₁₂₋₁₅ Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E Acetate | Tocopheryl Acetate |
| B | 4.00 | Z-COTE MAX | Zinc Oxide (and) Diphenyl |
| | | | Capryl Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1.50 | Veegum Ultra | Magnesium Aluminum Silicate |
| | 49.45 | Water dem. | Demineralized Water |

1.0% Peroxide decomposer

| | | |
|---|---|---|
| D | q.s. | Preservative |

| | | | |
|---|---|---|---|
| A | 8.00 | Cetiol B | Dibutyl Adipate |
| | 8.00 | Finsolv TN | C₁₂₋₁₅ Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E Acetate | Tocopheryl Acetate |
| B | 4.00 | T-Lite SF | Titanium Dioxide, Alumina |
| | | | Hydrate, Dimethicone/Methicone |
| | | | Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1.50 | Veegum Ultra | Magnesium Aluminum Silicate |
| | 50.25 | Water dem. | Demineralized Water |
| D | 0.50 | Citric Acid | Citric Acid |

q.s. Perfume Oil
0.2% Peroxide decomposer
q.s. Preservative

| | | | |
|---|---|---|---|
| A | 5.00 | Cosmacol EMI | Di-C₁₂₋₁₃ Alkyl Malate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 2.50 | T-Lite SF | Titanium Dioxide, Alumina |
| | | | Hydrate, |
| | | | Dimethicone/Methicone |
| | | | Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 42.00 | Water dem. | Aqua dem |
| D | 5.00 | Eusolex 232 | Phenylbenzimidazole Sulfonic |
| | | | Acid |
| | 10.00 | Water dem. | Aqua dem |
| E | 0.70 | Sodium Hydroxide | Sodium Hydroxide |

1.0% Peroxide decomposer
q.s. Preservative

| | | | |
|---|---|---|---|
| A | 5.00 | Cosmacol EMI | Di-C₁₂₋₁₃ Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl |
| | | | Hexyl Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 2.50 | T-Lite SF | Titanium Dioxide, Alumina |
| | | | Hydrate, |
| | | | Dimethicone/Methicone |
| | | | Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 41.00 | Water dem. | Aqua dem |
| D | 5.00 | Eusolex 232 | Phenylbenzimidazole Sulfonic |
| | | | Acid |

2.0% Peroxide decomposer

| | | | |
|---|---|---|---|
| | 10.00 | Water dem. | Aqua dem |
| E | 0.70 | Sodium Hydroxide | Sodium Hydroxide |
| | q.s. | Preservative | |

| | | | |
|---|---|---|---|
| A | 5.00 | Cosmacol EMI | Di-C₁₂₋₁₃ Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl |
| | | | Hexyl Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 2.50 | Z-COTE MAX | Zinc Oxide (and) Diphenyl |
| | | | Capryl Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 42.50 | Water dem. | Aqua dem |
| D | 5.00 | Eusolex 232 | Phenylbenzimidazole Sulfonic |
| | | | Acid |

0.5% Peroxide decomposer

| | | | |
|---|---|---|---|
| | 10.00 | Water dem. | Aqua dem |
| E | 0.70 | Sodium Hydroxide | Sodium Hydroxide |

q.s. Preservative

| | | | |
|---|---|---|---|
| A | 5.00 | Cosmacol EMI | Di-C₁₂₋₁₃ Alkyl Malate |
| | 7.50 | Uvinul N 539 T | Octocrylene |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 5.00 | Z-COTE MAX | Zinc Oxide (and) Diphenyl |
| | | | Capryl Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 40.50 | Water dem. | Aqua dem |
| D | 5.00 | Eusolex 232 | Phenylbenzimidazole Sulfonic |
| | | | Acid |

1.0% Peroxide decomposer

| | | | |
|---|---|---|---|
| | 10.00 | Water dem. | Aqua dem |
| E | 0.70 | Sodium Hydroxide | Sodium Hydroxide |

q.s. Preservative

| | | | |
|---|---|---|---|
| A | 5.00 | Cosmacol EMI | Di-C₁₂₋₁₃ Alkyl Malate |
| | 5.50 | Uvinul N 539 T | Octocrylene |
| | 2.00 | Uvinul A Plus | Diethylamino Hydrobenzoyl |
| | | | Hexyl Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 5.00 | Z-COTE MAX | Zinc Oxide (and) Diphenyl |
| | | | Capryl Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 41.50 | Water dem. | Aqua dem |
| D | 5.00 | Eusolex 232 | Phenylbenzimidazole Sulfonic |
| | | | Acid |

0.5% Peroxide decomposer

| | | | |
|---|---|---|---|
| | 9.50 | Water dem. | Aqua dem |
| E | 0.70 | Sodium Hydroxide | Sodium Hydroxide |

q.s. Preservative

| | | | |
|---|---|---|---|
| A | 5.00 | Cosmacol EMI | Di-C₁₂₋₁₃ Alkyl Malate |
| | 5.50 | Uvinul N 539 T | Octocrylene |
| | 2.00 | Uvinul A Plus | Diethylamino Hydrobenzoyl |
| | | | Hexyl Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E Acetate | Tocopheryl Acetate |
| | 1.00 | Ganex V-220 | VP/Eicosene Copolymer |
| | 5.00 | T-Lite SF-S | Titanium Dioxide, Silica |
| | | | Hydrate, Alumina Hydrate, |
| | | | Methicone/Dimethicone |
| | | | Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 40.50 | Water dem. | Aqua dem |
| D | 5.00 | Eusolex 232 | Phenylbenzimidazole Sulfonic |
| | | | Acid |

1.0% Peroxide decomposer

| | | | |
|---|---|---|---|
| | 10.00 | Water dem. | Aqua dem |
| E | 0.70 | Sodium Hydroxide | Sodium Hydroxide |

q.s. Preservative

| | | | |
|---|---|---|---|
| A | 8.00 | Cetiol B | Dibutyl Adipate |
| | 8.00 | Finsolv TN | C₁₂₋₁₅ Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl Triazine |
| | 1.00 | Uvinul T 150 | Ethylhexyl Triazone |
| | 1.00 | Vitamin E Acetate | Tocopheryl Acetate |
| B | 5.00 | T-Lite SF | Titanium Dioxide, Alumina |
| | | | Hydrate, |
| | | | Dimethicone/Methicone |
| | | | Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |

2.0% Peroxide decomposer

| | | | |
|---|---|---|---|
| | 1.50 | Veegum Ultra | Magnesium Aluminum Silicate |
| | ad 100 | Water dem. | Aqua dem |
| D | q.s. | Perfume-Oil | |

q.s. Preservative

| | | | |
|---|---|---|---|
| A | 8.00 | Cetiol B | Dibutyl Adipate |
| | 8.00 | Finsolv TN | C₁₂₋₁₅ Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl triazine |
| | 1.00 | Uvinul T 150 | Ethylhexyl Triazone |
| | 1.00 | Vitamin E Acetate | Tocopheryl Acetate |
| B | 5.00 | Z-COTE MAX | Zinc Oxide (and) Diphenyl |
| | | | Capryl Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |

1.0% Peroxide decomposer

| | | | |
|---|---|---|---|
| | 1.50 | Veegum Ultra | Magnesium Aluminum Silicate |
| | ad 100 | Water dem. | Aqua dem |
| D | q.s. | Perfume Oil | |

q.s. Preservative

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 57.00 | Water dem. | Demineralized Water |
| B | 5.00 | T-Lite SF-S | Titanium Dioxide, Silica |
| | | | Hydrate, Alumina Hydrate, |
| | | | Methicone/Dimethicone |
| | | | Copolymer |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 Fluid | Cyclopentasiloxane, |
| | | | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Bienenwachs 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |

2.0% Peroxide decomposer

| | | |
|---|---|---|
| 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 58.00 | Water dem. | Demineralized Water |
| B | 5.00 | Z-COTE MAX | Zinc Oxide (and) Diphenyl |
| | | | Capryl Methicone |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 Fluid | Cyclopentasiloxane, |
| | | | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Bienenwachs 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |

1.0% Peroxide decomposer

| | | |
|---|---|---|
| 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 58.50 | Water dem. | Demineralized Water |
| B | 5.00 | T-Lite SF-S | Titanium Dioxide, Silica |
| | | | Hydrate, Alumina Hydrate, |
| | | | Methicone/Dimethicone |
| | | | Copolymer |
| C | 7.50 | Uvinul MC 80 | Ethylhexyl Methoxycinnamate |
| | 5.00 | Uvinul N 539 T | Octocrylene |
| | 2.00 | Parsol 1789 | Butyl |
| | | | Methoxydibenzoylmethane |
| | 2.00 | Dow Corning 345 Fluid | Cyclopentasiloxane, |
| | | | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Bienenwachs 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |

0.5% Peroxide decomposer

| | | |
|---|---|---|
| 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Mexoryl SL | Terephthalidene Dicamphor |
| | | | Sulfonic Acid |
| | 58.40 | Water dem. | Demineralized Water |
| B | 6.00 | T-Lite SF-S | Titanium Dioxide, Silica |
| | | | Hydrate, Alumina Hydrate, |
| | | | Methicone/Dimethicone |
| | | | Copolymer |
| | 6.00 | Uvinul N 539 T | Octocrylene |
| | 2.00 | Parsol 1789 | Butyl |
| | | | Methoxydibenzoylmethane |
| | 2.00 | Dow Corning 345 Fluid | Cyclopentasiloxane, |
| | | | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Bienenwachs 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |

0.1% Peroxide decomposer

| | | |
|---|---|---|
| 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Mexoryl SL | Terephthalidene Dicamphor |
| | | | Sulfonic Acid |
| | 58.45 | Water dem. | Demineralized Water |
| B | 6.00 | Z-COTE MAX | Zinc Oxide (and) Diphenyl |
| | | | Capryl Methicone |
| | 6.00 | Uvinul N 539 T | Octocrylene |
| | 2.00 | Parsol 1789 | Butyl |
| | | | Methoxydibenzoylmethane |
| | 2.00 | Dow Corning 345 Fluid | Cyclopentasiloxane, |
| | | | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Bienenwachs 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |

0.05% Peroxide decomposer

| | | |
|---|---|---|
| 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Neoheliopan AP | Disodium Phenyl |
| | | | Dibenzimidazole Tetrasulfonate |
| | 57.50 | Water dem. | Demineralized Water |
| B | 6.00 | Z-COTE MAX | Zinc Oxide (and) Diphenyl |
| | | | Capryl Methicone |
| | 6.00 | Uvinul N 539 T | Octocrylene |
| | 2.00 | Parsol 1789 | Butyl |
| | | | Methoxydibenzoylmethane |
| | 2.00 | Dow Corning 345 Fluid | Cyclopentasiloxane, |
| | | | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Bienenwachs 3044 PH | Bees Wax |
| | 3.00 | Lanette O | Cetearyl Alcohol |
| | 10.00 | Miglyol 812 | Caprylic/Capric Triglyceride |
| D | 1.00 | Vitamin E Acetate | Tocopheryl Acetate |

1.0% Peroxide decomposer

| | | |
|---|---|---|
| 0.20 | Bisabolol rac. | Bisabolol |

| | | | |
|---|---|---|---|
| A | 25.00 | Dow Corning 345 Fluid | Cyclopentasiloxane, |
| | | | Cyclohexasiloxane |
| | 20.00 | Dow Corning 245 Fluid | Cyclopentasiloxane |
| | 8.00 | Uvinul MC 80 | Ethylhexyl Methoxycinnamate |
| | 4.00 | Abil EM 90 | Cetyl PEG/PPG-10/1 |
| | | | Dimethicone |
| | 7.00 | T-Lite SF | Titanium Dioxide, Alumina |
| | | | Hydrate, |
| | | | Dimethicone/Methicone |
| | | | Copolymer |
| B | 17.00 | Ethanol 95% | Alcohol |
| | 10.00 | Z-COTE MAX | Zinc Oxide (and) Diphenyl |
| | | | Capryl Methicone |
| | 4.50 | Water dem. | Demineralized Water |
| | 3.00 | Glycerin 87% | Glycerin |

0.5% Peroxide decomposer

| | | |
|---|---|---|
| 1.00 | Talc | Talc |

| | | | |
|---|---|---|---|
| A | 25.00 | Dow Corning 345 Fluid | Cyclopentasiloxane, |
| | | | Cyclohexasiloxane |
| | 20.00 | Dow Corning 245 Fluid | Cyclopentasiloxane |
| | 8.00 | Uvinul MC 80 | Ethylhexyl Methoxycinnamate |
| | 4.00 | Abil EM 90 | Cetyl PEG/PPG-10/1 |
| | | | Dimethicone |
| | 7.00 | T-Lite SF-S | Titanium Dioxide, Silica |
| | | | Hydrate, Alumina Hydrate, |
| | | | Methicone/Dimethicone |
| | | | Copolymer |
| B | 17.00 | Ethanol 95% | Alcohol |
| | 9.99 | Z-COTE MAX | Zinc Oxide (and) Diphenyl |
| | | | Capryl Methicone |
| | 5.00 | Water dem. | Demineralized Water |
| | 3.00 | Glycerin 87% | Glycerin |

0.01% Peroxide decomposer

| | | |
|---|---|---|
| 1.00 | Talc | Talc |

| | | | |
|---|---|---|---|
| A | 20.00 | Dow Corning 345 Fluid | Cyclopentasiloxane, |
| | | | Cyclohexasiloxane |
| | 10.00 | Dow Corning 245 Fluid | Cyclopentasiloxane |
| | 7.50 | Uvinul MC 80 | Ethylhexyl Methoxycinnamate |
| | 2.00 | Uvinul T 150 | Ethylhexyl Triazone |
| | 12.00 | Cosmacol EMI | Di-C₁₂₋₁₃ Alkyl Malate |
| | 4.00 | Abil EM 90 | Cetyl PEG/PPG-10/1 |
| | | | Dimethicone |
| B | 7.00 | T-Lite SF | Titanium Dioxide, Alumina |
| | | | Hydrate, |
| | | | Dimethicone/Methicone |
| | | | Copolymer |
| C | 15.00 | Ethanol 95% | Alcohol |
| | 10.00 | Z-COTE MAX | Zinc Oxide (and) Diphenyl |
| | | | Capryl Methicone |
| | 3.00 | Glycerin 87% | Glycerin |

1.5% Peroxide decomposer

| | | |
|---|---|---|
| 8.00 | Water dem. | Demineralized Water |

| | | | |
|---|---|---|---|
| A | 20.00 | Dow Corning 345 Fluid | Cyclopentasiloxane, |
| | | | Cyclohexasiloxane |
| | 10.00 | Dow Corning 245 Fluid | Cyclopentasiloxane |
| | 7.50 | Uvinul MC 80 | Ethylhexyl Methoxycinnamate |
| | 2.00 | Tinosorb S | Bis-Ethylhexyloxyphenol |
| | | | Methoxyphenyl Triazine |
| | 12.00 | Cosmacol EMI | Di-C₁₂₋₁₃ Alkyl Malate |
| | 4.00 | Abil EM 90 | Cetyl PEG/PPG-10/1 |
| | | | Dimethicone |
| B | 7.00 | T-Lite SF | Titanium Dioxide, Alumina |
| | | | Hydrate, |
| | | | Dimethicone/Methicone |
| | | | Copolymer |
| C | 15.00 | Ethanol 95% | Alcohol |
| | 10.00 | Z-COTE MAX | Zinc Oxide (and) Diphenyl |
| | | | Capryl Methicone |
| | 3.00 | Glycerin 87% | Glycerin |

0.5% Peroxide decomposer

| | | |
|---|---|---|
| 9.00 | Water dem. | Demineralized Water |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | T-Lite SF | Titanium Dioxide, Alumina |
| | | | Hydrate, |
| | | | Dimethicone/Methicone |
| | | | Copolymer |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, |
| | | | Hexyl Laurate |
| C | 0.50 | Sodium Chloride | Sodium Chloride |
| | 0.20 | Edeta BD | Disodium EDTA |
| | 61.30 | Water dem. | Aqua dem |
| D | 1.00 | Vitamin E Acetate | Tocopheryl Acetate |

1.0% Peroxide decomposer

| | |
|---|---|
| q.s. | Preservative |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl |
| | | | Capryl Methicone |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, |
| | | | Hexyl Laurate |
| C | 0.50 | Sodium Chloride | Sodium Chloride |
| | 0.20 | Edeta BD | Disodium EDTA |
| | 62.20 | Water dem. | Aqua dem |
| D | 1.00 | Vitamin E Acetate | Tocopheryl Acetate |

0.1% Peroxide decomposer

| | |
|---|---|
| q.s. | Preservative |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl |
| | | | Capryl Methicone |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Tinosorb S | Bis-Ethylhexyloxyphenol |
| | | | Methoxyphenyl Triazine |
| | 7.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, |
| | | | Hexyl Laurate |
| C | 0.50 | Sodium Chloride | Sodium Chloride |
| | 0.20 | Edeta BD | Disodium EDTA |
| | 60.30 | Water dem. | Aqua dem |
| D | 1.00 | Vitamin E Acetate | Tocopheryl Acetate |

2.0% Peroxide decomposer

| | |
|---|---|
| q.s. | Preservative |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and) Diphenyl |
| | | | Capryl Methicone |
| | 4.00 | Isopropyl Palmitate | Isopropyl Palmitate |
| | 1.00 | Abil 350 | Dimethicone |
| B | 4.00 | Uvinul N 539 T | Octocrylene |
| | 1.00 | Tinosorb S | Bis-Ethylhexyloxyphenol |
| | | | Methoxyphenyl Triazine |
| | 7.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, |
| | | | Hexyl Laurate |
| C | 0.50 | Sodium Chloride | Sodium Chloride |
| | 0.20 | Edeta BD | Disodium EDTA |
| | 62.10 | Water dem. | Aqua dem |
| D | 1.00 | Vitamin E Acetate | Tocopheryl Acetate |

0.2% Peroxide decomposer

| | |
|---|---|
| q.s. | Preservative |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and) Diphenyl |
| | | | Capryl Methicone |
| | 4.00 | Isopropyl Palmitate | Isopropyl Palmitate |
| | 1.00 | Abil 350 | Dimethicone |
| B | 3.00 | Uvinul N 539 T | Octocrylene |
| | 1.00 | Mexoryl XL | Drometrizole Trisiloxane |
| | 1.00 | Tinosorb S | Bis-Ethylhexyloxyphenol |
| | | | Methoxyphenyl Triazine |
| | 7.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, |
| | | | Hexyl Laurate |
| C | 0.50 | Sodium Chloride | Sodium Chloride |
| | 0.20 | Edeta BD | Disodium EDTA |

1.0% Peroxide decomposer

| | | | |
|---|---|---|---|
| | 61.30 | Water dem. | Aqua dem |
| D | 1.00 | Vitamin E Acetate | Tocopheryl Acetate |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | T-Lite SF-S | Titanium Dioxide, Silica |
| | | | Hydrate, Alumina Hydrate, |
| | | | Methicone/Dimethicone |
| | | | Copolymer |
| | 4.00 | Isopropyl Palmitate | Isopropyl Palmitate |
| | 1.00 | Abil 350 | Dimethicone |
| B | 3.00 | Uvinul N 539 T | Octocrylene |
| | 1.00 | Mexoryl XL | Drometrizole trisiloxane |
| | 1.00 | Tinosorb S | Bis-Ethylhexyloxyphenol |
| | | | Methoxyphenyl Triazine |
| | 7.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, |
| | | | Hexyl Laurate |
| C | 0.50 | Sodium Chloride | Sodium Chloride |
| | 0.20 | Edeta BD | Disodium EDTA |
| | 61.80 | Water dem. | Aqua dem |
| D | 1.00 | Vitamin E Acetate | Tocopheryl Acetate |

0.5% Peroxide decomposer

| | |
|---|---|
| q.s. | Preservative |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Uvinul T 150 | Ethylhexyl Triazone |
| | 2.50 | Cosmacol EMI | Di-C₁₂₋₁₃ Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C₁₂₋₁₃ Alkyl Malate |
| | 5.00 | Z-COTE MAX | Zinc Oxide (and) Diphenyl |
| | | | Capryl Methicone |
| D | 44.00 | Water dem. | Demineralized Water |
| | 5.00 | Glycerin 87% | Glycerin |
| | 1.00 | Lanette E | Sodium Cetearyl Sulfate |
| | 15.00 | Luviquat Care | Polyquaternium-44 |

1.0% Peroxide decomposer

| | | | |
|---|---|---|---|
| | 0.50 | Keltrol | Xanthan Gum |
| E | 1.00 | Phenonip | |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Tinosorb S | Bis-Ethoxylhexyloxyphenol |
| | | | Methoxyphenyl Triazine |
| | 2.50 | Cosmacol EMI | Di-C₁₂₋₁₃ Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C₁₂₋₁₃ Alkyl Malate |
| | 5.00 | Z-COTE MAX | Zinc Oxide (and) Diphenyl |
| | | | Capryl Methicone |
| D | 44.90 | Water dem. | Demineralized Water. |
| | 5.00 | Glycerin 87% | Glycerin |
| | 1.00 | Lanette E | Sodium Cetearyl Sulfate |
| | 15.00 | Luviquat Care | Polyquaternium-44 |

0.1% Peroxide decomposer

| | | | |
|---|---|---|---|
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C₁₂₋₁₃ Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C₁₂₋₁₃ Alkyl Malate |
| | 5.00 | Z-COTE MAX | Zinc Oxide (and) Diphenyl |
| | | | Capryl Methicone |
| D | 44.50 | Water dem. | Demineralized Water |
| | 5.00 | Glycerin 87% | Glycerin |
| | 1.00 | Lanette E | Sodium Cetearyl Sulfate |
| | 15.00 | Luviquat Care | Polyquaternium-44 |

1.5% Peroxide decomposer

| | | | |
|---|---|---|---|
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C₁₂₋₁₃ Alkyl Malate |
| B | 0.50 | Vitamin E Acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C₁₂₋₁₃ Alkyl Malate |
| | 5.00 | T-Lite SF | Titanium Dioxide, Alumina |
| | | | Hydrate, |
| | | | Dimethicone/Methicone |
| | | | Copolymer |
| D | 45.00 | Water dem. | Demineralized Water |
| | 5.00 | Glycerin 87% | Glycerin |
| | 1.00 | Lanette E | Sodium Cetearyl Sulfate |
| | 15.00 | Luviquat Care | Polyquaternium-44 |

1.0% Peroxide decomposer

| | | | |
|---|---|---|---|
| | 0.50 | Keltrol | Xanthan Gum |
| E | 1.00 | Phenonip | |

| | | | |
|---|---|---|---|
| A | 1.00 | Abil Care 85 | Bis-PEG/PPG-16/16 PEG/PPG-16/ |
| | | | 16 Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 5.00 | Uvinul MC 80 | Ethylhexyl Methoxycinnamate |
| | 10.00 | Witconol APM | PPG-3 Myristyl Ether |
| | 2.00 | Uvinul T 150 | Ethylhexyl Triazone |
| | 1.00 | Dow Corning 345 Fluid | Cyclopentasiloxane, |
| | | | Cyclohexasiloxane |
| | 5.00 | Uvinul N 539 T | Octocrylene |
| B | 5.00 | Z-COTE MAX | Zinc Oxide (and) Diphenyl Capryl |
| | | | Methicone |
| C | 5.00 | 1,2-Propylene Glycol | Propylene Glycol |
| | 0.20 | Keltrol | Xanthan Gum |
| | 0.10 | Edeta BD | Disodium EDTA |

2.0% Peroxide decomposer

| | | | |
|---|---|---|---|
| | 1.50 | Simulgel 600 | Acrylamide/Sodium |
| | | | Acryloyldimethyltaurate |
| | | | Copolymer, Isohexadecane, |
| | | | Polysorbate 80 |
| | 58.40 | Water dem. | Demineralized Water |
| D | q.s. | Perfume Oil | |
| | 0.50 | Glydant | DMDM Hydantoin |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C₁₂₋₁₅ Alkyl Benzoate |
| | 1.50 | Miglyol 812 | Caprylic/Capric Triglyceride |
| | 0.50 | Vitamin E Acetate | Tocopheryl Acetate |
| | 7.50 | Uvinul MC 80 | Ethylhexyl Methoxycinnamate |
| | 4.00 | Cetiol B | Dibutyl Adipate |
| | 3.00 | Luvitol EHO | Cetearyl Ethylhexanoate |
| | 1.00 | Cremophor CO 40 | PEG-40 Hydrogenated Castor Oil |
| | 1.00 | Paraffin Oil, low | Mineral Oil |
| | | viscosity | |
| | 3.00 | Plantacare 2000 | Decyl Glucoside |
| | 0.50 | Phenonip | |
| | q.s. | Perfume Oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate Copolymer, |
| | | | Squalane, Polysorbate 60 |
| | 0.20 | Keltrol | Xanthan Gum |
| | 0.10 | Edeta BD | Disodium EDTA |

1.0% Peroxide decomposer

| | | |
|---|---|---|
| 64.20 | Water dem. | Aqua dem |

| | | | |
|---|---|---|---|
| A | 6.00 | Gilugel SIL 5 | Cyclomethicone (and) |
| | | | Aluminium/Magnesium Hydroxide |
| | | | Stearate |
| | 5.00 | Uvinul MC 80 | Ethylhexyl Methoxycinnamate |
| | 2.00 | Uvinul A Plus | Diethylamino Hydroxybenzoyl Hexyl |
| | | | Benzoate |
| | 1.00 | Uvinul T 150 | Ethylhexyl Triazone |
| | 7.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| | 2.00 | Cosmacol EMI | Di-C₁₂₋₁₃ Alkyl Malate |
| | 3.00 | Isopropyl Palmitate | Isopropyl Palmitate |
| | 5.00 | Abil B 8839 | Cyclopentasiloxane, Cyclohexasiloxane |
| | 0.50 | Abil 350 | Dimethicone |
| B | 0.50 | Sodium Chloride | Sodium Chloride |
| | 0.20 | Edeta BD | Disodium EDTA |

0.2% Peroxide decomposer

| | | | |
|---|---|---|---|
| | 62.10 | Water dem. | Demineralized Water |
| C | 1.00 | Vitamin E Acetate | Tocopheryl Acetate |
| | 0.50 | Phenonip | |

| | | | |
|---|---|---|---|
| A | 4.50 | Uvinul MC 80 | Ethylhexyl Methoxycinnamate |
| | 2.00 | Tinosorb S | Bis-Ethylhexyloxyphenol |
| | | | Methoxyphenyl |
| | | | Triazine |
| | 3.00 | Uvinul N 539 T | Octocrylene |
| | 2.50 | Cosmacol EMI | Di-C₁₂₋₁₃ Alkyl Malate |
| | 0.50 | Vitamin E Acetate | Tocopheryl Acetate |
| | 4.00 | Tego Care 450 | Polyglyceryl-3 Methyl Glucose |
| | | | Distearate |
| B | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C₁₂₋₁₃ Alkyl Malate |
| | 3.00 | T-Lite SF | Titanium Dioxide, Alumina Hydrate, |
| | | | Dimethicone/Methicone Copolymer |
| C | 5.00 | Glycerin 87% | Glycerin |
| | 1.00 | Lanette E | Sodium Cetearyl Sulfate |
| | 0.50 | Keltrol | Xanthan Gum |

1.0% Peroxide decomposer

| | | | |
|---|---|---|---|
| | 59.70 | Water dem. | Demineralized Water |
| D | 1.00 | Phenonip | |
| | 0.30 | Bisabolol rac. | Bisabolol |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C₁₂₋₁₅ Alkyl Benzoate |
| | 1.50 | Miglyol 812 | Caprylic/Capric Triglyceride |
| | 0.50 | Vitamin E Acetate | Tocopheryl Acetate |
| | 7.50 | Uvinul MC 80 | Ethylhexyl Methoxycinnamate |
| | 4.00 | Cetiol B | Dibutyl Adipate |
| | 1.00 | Luvitol EHO | Cetearyl Ethylhexanoate |
| | 1.00 | Cremophor CO 40 | PEG-40 Hydrogenated Castor Oil |
| | 1.00 | Paraffin Oil, low | Mineral Oil |
| | | viscosity | |
| | 3.00 | Plantacare 2000 | Decyl Glucoside |
| | 1.00 | Uvinul A Plus | Diethylamino Hydroxybenzoyl Hexyl |
| | | | Benzoate |
| | 0.50 | Phenonip | |
| | 2.50 | Uvinul T 150 | Ethylhexyl Triazone |
| | q.s. | Perfume Oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate Copolymer, |
| | | | Squalane, Polysorbate 60 |
| | 0.10 | Keltrol | Xanthan Gum |
| | 0.10 | Edeta BD | Disodium EDTA |

0.5% Peroxide decomposer

| | | |
|---|---|---|
| 59.90 | Water dem. | Demineralized Water |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C₁₂₋₁₅ Alkyl Benzoate |
| | 8.00 | Cetiol B | Dibutyl Adipate |
| B | 6.00 | T-Lite SF | Titanium Dioxide, Alumina Hydrate, |
| | | | Dimethicone/Methicone Copolymer |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |

2.0% Peroxide decomposer

| | | | |
|---|---|---|---|
| | 0.30 | Keltrol | Xanthan Gum |
| | 45.10 | Water dem. | Water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C₁₂₋₁₅ Alkyl Benzoate |
| | 8.00 | Cetiol B | Dibutyl Adipate |
| B | 6.00 | Uvinul N 539 T | Octocrylene |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 0.30 | Carbopol Ultrez 10 P | Carbomer |

1.0% Peroxide decomposer

| | | | |
|---|---|---|---|
| | 46.10 | Water dem. | Demineralized water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 10.00 | Myritol 331 | Cocoglycerides |
| | 8.00 | Finsolv TN | C₁₂₋₁₅ Alkyl Benzoate |
| | 8.00 | Cetiol B | Dibutyl Adipate |
| B | 2.00 | Uvinul A Plus | Diethylamino Hydroxybenzoyl Hexyl |
| | | | Benzoate |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |

5.0% Peroxide decomposer

| | 0.30 | Carbopol Ultrez 10 P | Carbomer |
|---|---|---|---|
| | ad Water dem. | | Water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 3.50 | Cremophor A 6 | Ceteareth-6, Stearyl Alcohol |
| | 1.50 | Cremophor A 25 | Ceteareth-25 |
| | 7.50 | Uvinul MC 80 | Ethylhexyl Methoxycinnamate |
| | 2.00 | Uvinul A Plus | Diethylamino Hydroxybenzoyl Hexyl |
| | | | Benzoate |
| | 2.00 | Dow Corning 345 | Cyclopentasiloxane, |
| | | | Cyclohexasiloxane |
| | | | Fluid |
| | 0.50 | Bienenwachs 3044 PH | Bees Wax |
| | 3.00 | Lanette O | Cetearyl Alcohol |
| | 10.00 | Miglyol 812 | Caprylic/Capric Triglyceride |
| B | 5.00 | T-Lite SF-S Titanium | Titanium Dioxide, Silica |
| | | | Hydrate, Alumina |
| | | | Hydrate, Methicone/Dimethicone |
| | | | Copolymer |
| C | 3.00 | Glycerin 87% | Glycerin |
| | 0.20 | Edeta BD | Disodium EDTA |
| | 0.30 | Keltrol T | Xanthan Gum |
| | 1.00 | Plantacare 2000 | Decyl Glucoside |

2.0% Peroxide decomposer

| | | | |
|---|---|---|---|
| | 57.30 | Water dem. | Demineralized Water |
| D | 1.00 | Vitamin E Acetate | Tocopheryl Acetate |
| | 0.20 | Bisabolol rac. | Bisabolol |

| | | | |
|---|---|---|---|
| A | 10.00 | Uvinul A Plus B | Ethylhexyl Methoxycinnamate, |
| | | | Diethylamino Hydroxybenzoyl Hexyl |
| | | | Benzoate |
| | 10.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C₁₂₋₁₅ Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 3.00 | Z-COTE MAX | Zinc Oxide (and) Diphenyl Capryl |
| | | | Methicone |
| C | 35.08 | Water dem. | Demineralized Water |
| | 0.38 | Citric Acid | Citric Acid |
| | 2.9 | Glycerin 87% | Glycerin |
| | 0.05 | Edeta BD | Disodium EDTA |
| | 0.20 | Allantoin | Allantoin |
| | 0.30 | Keltrol | Xanthan Gum |

0.1% Peroxide decomposer

| | | | |
|---|---|---|---|
| | 1.50 | Veegum Ultra | Magnesium Aluminum Silicate |
| D | 0.50 | Phenonip | |
| | 1.00 | Vitamin E Acetate | Tocopheryl Acetate |

| | | | |
|---|---|---|---|
| A | 0.70 | Cremophor A 25 | Ceteareth-25 |
| | 1.70 | Cremophor A 6 | Ceteareth-6, Stearyl Alcohol |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Z-COTE MAX | Zinc Oxide (and) Diphenyl Capryl |
| | | | Methicone |
| | 2.00 | Abil B 8843 | PEG-14 Dimethicone |
| | 3.60 | Lanette O | Cetearyl Alcohol |
| | 4.00 | Uvinul MC 80 | Ethylhexyl Methoxycinnamate |
| | 2.00 | Cetiol B | Dibutyl Adipate |
| B | 4.00 | Glycerin 87% | Glycerin |
| | 0.20 | Edeta BD | Disodium EDTA |
| | 71.00 | Water dem. | Panthenol |
| C | 4.00 | Luvigel EM | Caprylic/Capric Triglyceride, Sodium |
| | | | Acrylates Copolymer |
| D | 1.00 | Vitamin E Acetate | Tocopheryl Acetate |

1.0% Peroxide decomposer

| | | |
|---|---|---|
| 0.20 | Bisabolol rac. | Bisabolol |
| q.s. | Preservative | |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl |
| | | | Methicone |
| | 0.03 | Sicomet Blau P | C.I. 77 007, Ultramarines |
| | | 77 007 | |
| C | 4.00 | 1,2-Propylene | Propylene Glycol |
| | | Glycol Care | |
| | 2.00 | D-Panthenol 50 P | Panthenol, Propylene Glycol |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |

0.5% Peroxide decomposer

| | | |
|---|---|---|
| 65.27 | Water dem. | Demineralized Water |
| q.s. | Preservative | |
| 0.50 | Vitamin E Acetate | Tocopheryl Acetate |

| | | | |
|---|---|---|---|
| A | 6.00 | Gilugel SIL 5 | Cyclomethicone (and) |
| | | | Aluminium/Magnesium Hydroxide |
| | | | Stearate |
| | 5.00 | Uvinul N 539 T | Octocrylene |
| | 2.00 | Mexoryl XL | Drometrizole Trisiloxane |
| | 1.00 | Uvinul T 150 | Ethylhexyl Triazone |
| | 3.00 | T-Lite SF-S | Titanium Dioxide, Silica Hydrate, |
| | | | Alumina |
| | | | Hydrate, Methicone/Dimethicone |
| | | | Copolymer |
| | 5.00 | Finsolv TN | C₁₂₋₁₅ Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| | 2.00 | Cosmacol EMI | Di-C₁₂₋₁₃ Alkyl Malate |
| | 3.00 | Isopropyl Palmitate | Isopropyl Palmitate |
| | 5.00 | Abil B 8839 | Cyclopentasiloxane, Cyclohexasiloxane |
| | 0.50 | Abil 350 | Dimethicone |
| B | 0.50 | Sodium Chloride | Sodium Chloride |
| | 0.20 | Edeta BD | Disodium EDTA |

1.0% Peroxide decomposer

| | | | |
|---|---|---|---|
| | 61.30 | Water dem. | Demineralized Water |
| C | 1.00 | Vitamin E Acetate | Tocopheryl Acetate |
| | 0.50 | Phenonip | |

| | | | |
|---|---|---|---|
| A | 1.00 | Abil Care 85 | Bis-PEG/PPG-16/16 PEG/PPG-16/16 |
| | | | Dimethicone, Caprylic/Capric Triglyceride |
| | 3.00 | Cremophor | PEG-40 Hydrogenated Castor Oil |
| | | CO 40 | |
| | 0.30 | Cremophor | PEG-7 Hydrogenated Castor Oil |
| | | WO 7 | |
| | 5.00 | Uvinul | Octocrylene |
| | | N 539 T | |
| | 10.00 | Witconol APM | PPG-3 Myristyl Ether |
| | 2.00 | Uvinul T 150 | Ethylhexyl Triazone |
| | 1.00 | Dow Corning | Cyclopentasiloxane, Cyclohexasiloxane |
| | | 345 | |
| | | Fluid | |
| | 2.00 | Uvinul A Plus | Diethylamino Hydroxybenzoyl Hexyl |
| | | | Benzoate |
| B | 5.00 | Z-COTE MAX | Zinc Oxide (and) Diphenyl Capryl Silane |
| C | 5.00 | 1,2-Propylene | Propylene Glycol |
| | | Glycol | |
| | 0.20 | Keltrol | Xanthan Gum |
| | 0.10 | Edeta BD | Disodium EDTA |

1.0% Peroxide decomposer

| | | | |
|---|---|---|---|
| | 1.50 | Simulgel 600 | Acrylamide/Sodium |
| | | | Acryloyldimethyltaurate Copolymer, |
| | | | Isohexadecane, Polysorbate 80 |
| | 59.40 | Water dem. | Demineralized Water |
| D | q.s. | Perfume Oil | |
| | 0.50 | Glydant | DMDM Hydantoin |

| | | | |
|---|---|---|---|
| A | 7.00 | Uvinul A Plus B | Ethylhexyl Methoxycinnamate, |
| | | | Diethylamino Hydroxybenzoyl Hexyl |
| | | | Benzoate |
| | 1.00 | Tinosorb S | Bis-Ethylhexyloxyphenol |
| | | | Methoxyphenyl |
| | | | Triazine |
| | 1.00 | Uvinul T 150 | Ethylhexyl Triazone |
| | 7.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C₁₂₋₁₅ Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 5.00 | Z-COTE MAX | Zinc Oxide (and) Diphenyl Capryl |
| | | | Methicone |
| C | 35.35 | Water dem. | Demineralized Water |
| | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |

0.1% Peroxide decomposer

| | | | |
|---|---|---|---|
| | 1.50 | Veegum Ultra | Magnesium Aluminum Silicate |
| D | 0.50 | Phenonip | |
| | 1.00 | Vitamin E Acetate | Tocopheryl Acetate |

| | | | |
|---|---|---|---|
| A | 7.00 | Uvinul A Plus B | Ethylhexyl Methoxycinnamate, |
| | | | Diethylamino Hydroxybenzoyl Hexyl |
| | | | Benzoate |
| | 1.00 | Tinosorb S | Bis-Ethylhexyloxyphenol |
| | | | Methoxyphenyl |
| | | | Triazine |
| | 1.00 | Uvinul T 150 | Ethylhexyl Triazone |
| | 7.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C₁₂₋₁₅ Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 5.00 | T-Lite SF | Titanium Dioxide, Alumina Hydrate, |
| | | | Dimethicone/Methicone Copolymer |
| C | 30.45 | Water dem. | Demineralized Water |
| | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |

5.0% Peroxide decomposer

| | | | |
|---|---|---|---|
| | 1.50 | Veegum Ultra | Magnesium Aluminum Silicate |
| D | 0.50 | Phenonip | |
| | 1.00 | Vitamin E Acetate | Tocopheryl Acetate |

| | | |
|---|---|---|
| 5.00 | Z-COTE MAX | Zinc Oxide (and) Diphenyl Capryl |
| | | Methicone |
| 2.00 | Uvinul A Plus | Diethylamino Hydroxybenzoyl |
| | | Hexyl Benzoate |
| 2.00 | Mexoryl XL | Drometrizole Trisiloxane |
| 3.00 | Uvinul MC 80 | Ethylhexyl Methoxycinnamate |
| 0.50 | Abil 350 | Dimethicone |
| 2.75 | Carnicowachs LT 20 | Carnauba (Copernica Cerifera) |
| | | Wax, Paraffin |
| 3.70 | Candelillawachs LT 281 LJ | Candelilla (Euphorbia Cerifera) Wax |
| 1.80 | Bienenwachs 3050 PH | Bees Wax |
| 3.20 | TeCero-Wachs 30445 | Microcrystalline Wax |
| 3.20 | TeCero-Wachs 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E Acetate | Tocopheryl Acetate |

1.0% Peroxide decomposer

| | | |
|---|---|---|
| 0.42 | D,L-Alpha-Tocopherol | Tocopherol |
| 40.38 | Ricinus Oil | Castor (Ricinus Communis) Oil |

| | | | |
|---|---|---|---|
| A | 1.00 | Abil Care 85 | Bis-PEG/PPG-16/16 PEG/PPG-16/ |
| | | | 16 Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 Fluid | Cyclopentasiloxane, |
| | | | Cyclohexasiloxane |
| | 5.00 | Uvinul N 539 T | Octocrylene |
| B | 3.00 | T-Lite SF-S | Titanium Dioxide, Silica |
| | | | Hydrate, Alumina Hydrate, |
| | | | Methicone/Dimethicone |
| | | | Copolymer |
| C | 5.00 | 1,2-Propylene Glycol | Propylene Glycol |
| | 1.00 | Mexoryl SX | Terephthalidene Dicamphor |
| | | | Sulfanic Acid |
| | 0.20 | Keltrol | Xanthan Gum |
| | 0.10 | Edeta BD | Disodium EDTA |

1.0% Peroxide decomposer

| | | | |
|---|---|---|---|
| | 1.50 | Simulgel 600 | Acrylamide/Sodium |
| | | | Acryloyldimethyltaurate |
| | | | Copolymer, Isohexadecane, |
| | | | Polysorbate 80 |
| | 69.50 | Water dem. | Demineralized Water |
| D | q.s. | Perfume Oil | |
| | 0.50 | Glydant | DMDM Hydantoin |

| | | | |
|---|---|---|---|
| A | 1.00 | Abil Care 85 | Bis-PEG/PPG-16/16 PEG/PPG-16/ |
| | | | 16 Dimethicone, Caprylic/Capric |
| | | | Triglyceride |
| | 3.00 | Cremophor CO 40 | PEG-40 Hydrogenated Castor |
| | | | Oil |
| | 0.30 | Cremophor WO 7 | PEG-7 Hydrogenated Castor |
| | | | Oil |
| | 2.00 | Parsol 1789 | Butyl |
| | | | Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 Fluid | Cyclopentasiloxane, |
| | | | Cyclohexasiloxane |
| | 5.00 | Uvinul N 539 T | Octocrylene |
| B | 3.00 | T-Lite SF-S | Titanium Dioxide, Silica Hydrate, |
| | | | Alumina Hydrate, Methicone/ |
| | | | Dimethicone Copolymer |
| C | 5.00 | 1,2-Propylene Glycol | Propylene Glycol |
| | 1.00 | Mexoryl SX | Terephthalidene Dicamphor |
| | | | Sulfanic Acid |
| | 0.20 | Keltrol | Xanthan Gum |
| | 0.10 | Edeta BD | Disodium EDTA |

0.2% Peroxide decomposer

| | | | |
|---|---|---|---|
| | 1.50 | Simulgel 600 | Acrylamide/Sodium |
| | | | Acryloyldimethyltaurate |
| | | | Copolymer, Isohexadecane, |
| | | | Polysorbate 80 |
| | 68.30 | Water dem. | Demineralized Water |
| D | q.s. | Perfume Oil | |
| | 0.50 | Glydant | DMDM Hydantoin |

| | | | |
|---|---|---|---|
| A | 0.70 | Cremophor A 25 | Ceteareth-25 |
| | 1.70 | Cremophor A 6 | Ceteareth-6, Stearyl Alcohol |
| | 2.00 | Parsol 1789 | Butyl Methoxydibenzoylmethane |
| | 5.00 | Uvinul N 539 T | Octocrylene |
| | 4.00 | Z-COTE MAX | Zinc Oxide (and) Diphenyl Capryl |
| | | | Methicone |
| | 2.00 | Abil B 8843 | PEG-14 Dimethicone |
| | 3.60 | Lanette O | Cetearyl Alcohol |
| | 1.00 | Uvinul T150 | Ethylhexyl Triazone |
| | 2.00 | Cetiol B | Dibutyl Adipate |
| B | 4.00 | Glycerin 87% | Glycerin |
| | 0.20 | Edeta BD | Disodium EDTA |
| | 71.00 | Water dem. | Panthenol |
| C | 4.00 | Luvigel EM | Caprylic/Capric Triglyceride, Sodium |
| | | | Acrylates Copolymer |

1.0% Peroxide decomposer

| | | | |
|---|---|---|---|
| D | 1.00 | Vitamin E Acetate | Tocopheryl Acetate |
| | 0.20 | Bisabolol rac. | Bisabolol |
| | q.s. | Preservative | |

| | | | |
|---|---|---|---|
| A | 1.00 | Abil Care 85 | Bis-PEG/PPG-16/16 PEG/PPG-16/ |
| | | | 16 Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl |
| | | | Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 Fluid | Cyclopentasiloxane, |
| | | | Cyclohexasiloxane |
| | 5.00 | Uvinul N 539 T | Octocrylene |
| B | 3.00 | Z-COTE MAX | Zinc Oxide (and) Diphenyl |
| | | | Capryl Methicone |
| C | 5.00 | 1,2-Propylene Glycol | Propylene Glycol |
| | 1.00 | Mexoryl SX | Terephthalidene Dicamphor |
| | | | Sulfanic Acid |
| | 0.20 | Keltrol | Xanthan Gum |
| | 0.10 | Edeta BD | Disodium EDTA |
| | 1.50 | Simulgel 600 | Acrylamide/Sodium |
| | | | Acryloyldimethyltaurate |
| | | | Copolymer, Isohexadecane, |
| | | | Polysorbate 80 |

0.5% Peroxide decomposer

| | | | |
|---|---|---|---|
| | 68.00 | Water dem. | Demineralized Water |
| D | q.s. | Perfume Oil | |
| | 0.50 | Glydant | DMDM Hydantoin |

| | | | |
|---|---|---|---|
| A | 2.00 | Uvinul A Plus | Diethylamino Hydroxybenzoyl |
| | | | Hexyl Benzoate |
| | 3.00 | Uvinul N 539 T | Octocrylene |
| | 3.00 | Emulgade PL 68/50 | Cetearyl Glucoside, Cetearyl |
| | | | Alcohol |
| | 2.00 | Dracorin 100 SE | Glyceryl Stearate, PEG-100 |
| | | | Stearate |
| | 1.00 | Fitoderm | Squalane |
| | 0.5 | Cremophor WO 7 | PEG-7 Hydrogenated Castor Oil |
| | 0.5 | Cremophor PS 20 | Polysorbate 20 |
| | 2.00 | Dry Flo Pure | Aluminium Starch |
| | | | Octenylsuccinate |
| B | 5.00 | Z-COTE MAX | Zinc Oxide (and) Diphenyl |
| | | | Capryl Methicone |
| C | 4.00 | 1,2-Propylene Glycol Care | Propylene Glycol |
| | 0.20 | Keltrol | Xanthan Gum |
| | 0.50 | Simulgel 600 | Acrylamide/Sodium |
| | | | Acryloyldimethyltaurate |
| | | | Copolymer, Isohexadecane, |
| | | | Polysorbate 80 |
| | 65.80 | Water dem. | Demineralized Water |
| D | 0.50 | Euxyl K 300 | |
| | 0.50 | Vitamin E Acetate | Tocopheryl Acetate |

1.0% Peroxide decomposer

| | | |
|---|---|---|
| 1.00 | RetiSTAR | Caprylic/Capric Triglyceride, |
| | | Sodium Ascorbate, |
| | | Tocopherol, Retinol |

1-8. (canceled)

9. A cosmetic or pharmaceutical agent for treating, comprising:
I) at least one peroxide decomposer of compounds of the general formulae I.1 to 1.3: 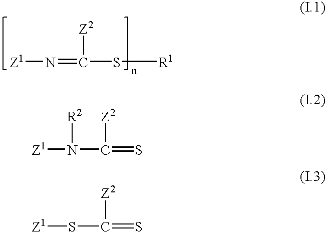
wherein
n is an integer from 1 to 3,
at least one of the radicals Z¹ and Z² is an organyl radical and the other is hydrogen or an organyl radical, wherein Z¹ and Z² may also together be a divalent bridging group having 1 to 5 atoms between the flanking bonds,
R¹ is hydrogen, alkyl, cycloalkyl, heterocycloalkyl, aryl, heteroaryl or an n-valent cation, and
R² is hydrogen, alkyl, cycloalkyl, heterocycloalkyl, aryl, hetaryl, COOR^{a}, COO⁻M⁺, SO₃R^{a}, SO₃⁻M⁺, sulfonamide, NE¹E², (NE¹E²E³)⁺A⁻, OR^{a}, SR^{a}, (CHR^{b}CH₂O)_{y}R^{a}, (CH₂O)_{y}R^{a}, (CH₂CH₂NE¹)_{y}R^{a}, alkylaminocarbonyl, dialkylaminocarbonyl, alkylcarbonylamino or acyl, wherein
R^{a} are in each case identical or different radicals selected from hydrogen, alkyl, cycloalkyl, aryl or hetaryl,
E¹, E², E³ are in each case identical or different radicals selected from hydrogen, alkyl, cycloalkyl, aryl or hetaryl,
R^{b} is hydrogen, methyl or ethyl,
M⁺ is a cation equivalent,
A⁻ is an anion equivalent and
y is an integer from 1 to 250;
II) if appropriate at least one antioxidant that is effective as free-radical scavenger; and
III) at least one cosmetically or pharmaceutically acceptable carrier.

10. The agent according to claim 9, comprising at least one tocopherol or tocopherol derivative.

11. The agent according to claim 9, wherein component III) is selected from
i) water,
ii) water-miscible organic solvents, preferably C₁-C₄-alkanols,
iii) oils, fats, waxes,
iv) esters of C₆-C₃₀-monocarboxylic acids with mono-, di- or trihydric alcohols that are different from iii),
v) saturated acyclic and cyclic hydrocarbons,
vi) fatty acids,
vii) fatty alcohols
and mixtures thereof

12. The agent according to claim 9, further comprising at least one component which is selected from cosmetically active ingredients, emulsifiers, surfactants, preservatives, perfume oils, thickeners, skin conditioners, photo-protective agents, bleaches, gel formers, care agents, colorants, tints, tanning agents, dyes, pigments, consistency regulators, humectants, refatting agents, collagen, protein hydrolysates, lipids, antifoams, antistats, emollients and softeners.

13. The agent according to claim 9 in the form of a gel, foam, spray, ointment, cream, emulsion, suspension, lotion, milk or paste.

14. The agent according to claim 9, which comprises component I) in an amount of from 0.0005 to 5% by weight, based on the total weight of the agent.

15. The agent according to claim 9, wherein the at least one peroxide decomposer is used in combination with at least one substance which absorbs UV rays in the UV-B and/or UV-A region.

16. The agent according to claim 9, for avoiding or reducing damage to the skin by peroxides and/or hydroperoxides formed as a result of endogenous or exogenous factors.

17. The agent according to claim 9, wherein the compounds of the formulae I.1 to I.3 are selected from 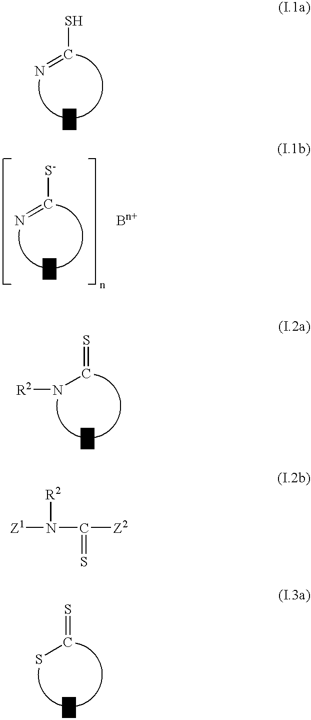
wherein
Q is a divalent bridging group having 2 to 5 atoms between the flanking bonds,
Bⁿ⁺ is a mono- to trivalent cation,
n is an integer from 1 to 3,
R² has the meanings given in claim 9,
Z¹ and Z², independently of one another, are hydrogen, alkyl, cycloalkyl, heterocycloalkyl, aryl, heteroaryl, amino, aminocarbonyl, aminothiocarbonyl or thiocarbamidyl, wherein at least one of the radicals Z¹ or Z² is aryl or heteroaryl.

18. The agent according to claim 9, wherein the compounds of the formulae I.1 to I.3 are selected from 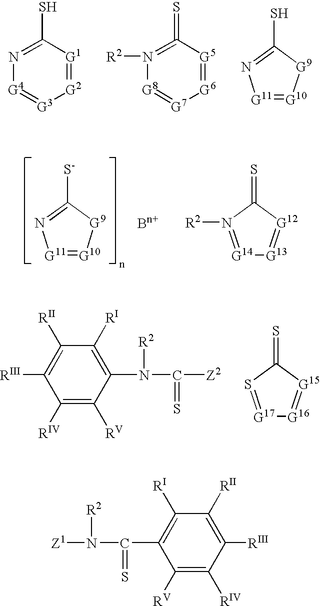
wherein
G¹ is N or CR³,
G² is N or CR⁴,
G³ is N or CR⁵,
G⁴ is N or CR⁶,
wherein
R³, R⁴, R⁵ and R⁶, independently of one another, are hydrogen, alkyl, cycloalkyl, heterocycloalkyl, aryl, heteroaryl, COOR^{c}, COO⁻M⁺, SO₃R^{c}, SO₃⁻M⁺, sulfonamide, NE⁴E⁵, (NE⁴E⁵E⁶)⁺A⁻, OR^{c}, SR^{c}, (CHR^{d}CH₂O)_{w}R^{c}, (CH₂O)_{w}R^{c}, (CH₂CH₂NE⁴)_{w}R^{c}, alkylaminocarbonyl, dialkylaminocarbonyl, alkylcarbonylamino, halogen, nitro, acyl or cyano, wherein
R^{c} are in each case identical or different radicals selected from hydrogen, alkyl, cycloalkyl, aryl or hetaryl,
E⁴, E⁵, E⁶ are in each case identical or different radicals selected from hydrogen, alkyl, cycloalkyl, aryl or hetaryl,
R^{d} is hydrogen, methyl or ethyl,
M⁺ is a cation equivalent,
A⁻ is an anion equivalent and
w is an integer from 1 to 250,
wherein two adjacent radicals R³ to R⁶ can also form an anellated ring system with one, two or three further rings,
G⁵ is BR⁷, CR⁷R⁸, SiR⁷R⁸, NR⁷, PR⁷, O or S,
G⁶ is BR⁹, CR⁹R¹⁰, SiR⁹R¹⁰, NR⁹, PR⁹, O or S,
G⁷ is BR¹¹, CR¹¹R¹², SiR¹¹R¹², NR¹¹, PR¹¹, O or S,
G⁸ is BR¹³, CR¹³R¹⁴, SiR¹³R¹⁴, NR¹³, PR¹³, O or S,
wherein
R⁷, R⁸, R⁹, R¹⁰, R¹¹, R¹², R¹³ and R¹⁴, independently of one another, can have the meanings given above for the radicals R³ to R⁶,
wherein two geminal radicals R⁷R⁸, R⁹R¹⁰, R¹¹R¹² and/or R¹³R¹⁴ can also together be a double-bonded oxygen atom or sulfur atom,
wherein two adjacent radicals R⁷, R⁹, R¹¹ and R¹³ can also be joined to anellated aliphatic or aromatic rings,
wherein two adjacent radicals R⁷, R⁹, R¹¹ and R¹³ can also together be the bond fraction of a double bond between the atoms to which they are bonded,
G⁹ is BR¹⁵, CR¹⁵R¹⁶, SiR¹⁵R¹⁶, NR¹⁵, PR¹⁵, O or S,
G¹⁰ is N or CR¹⁷,
G¹¹ is N or CR¹⁸,
wherein
R¹⁵, R¹⁶, R¹⁷ and R¹⁸, independently of one another, can have the meanings given above for the radicals R³ to R⁶,
wherein two adjacent radicals R¹⁷ and R¹⁸ can also form an anellated ring system with one, two or three further rings,
G¹² is BR¹⁹, CR¹⁹R²⁰, SiR¹⁹R²⁰, NR¹⁹, PR¹⁹, O or S,
G¹³ is BR²¹, CR²¹R²², SiR²¹R²², NR²¹, PR²¹, O or S,
G¹⁴ is BR²³, CR²³R²⁴, SiR²³R²⁴, NR²³, PR²³, O or S,
wherein
R¹⁹, R²⁰, R²¹, R²², R²³ and R²⁴, independently of one another, can have the meanings given above for the radicals R³ to R⁶,
wherein two geminal radicals R¹⁹R²⁰, R²¹R²² and/or R²³R²⁴ can also together be a double-bonded oxygen atom or sulfur atom,
wherein two adjacent radicals R¹⁹, R²¹ and R²³ can also be joined to anellated aliphatic or aromatic rings,
wherein two adjacent radicals R¹⁹, R²¹ and R²³ can also together be the bond fraction of a double bond between the atoms to which they are bonded,
G¹⁵ is BR²⁵, CR²⁵R²⁶, SiR²⁵R²⁶, NR²⁵, PR²⁵, O or S,
G¹⁶ is BR²⁷, CR²⁷R²⁸, SiR²⁷R²⁸, NR²⁷, PR²⁷, O or S,
G¹⁷ is BR²⁹, CR²⁹R³⁰, SiR²⁹R³⁰, NR²⁹, PR²⁹, O or S,
wherein
R²⁵, R²⁶, R²⁷, R²⁸, R²⁹ and R³⁰, independently of one another, can have the meanings given above for the radicals R³ to R⁶,
wherein two geminal radicals R²⁵R²⁶, R²⁷R²⁸ and/or R²⁹R³⁰ can also together be a double-bonded oxygen atom or sulfur atom,
wherein two adjacent radicals R²⁵, R²⁷ and R²⁹ can also be joined to anellated aliphatic or aromatic rings,
wherein two adjacent radicals R²⁵, R²⁷ and R²⁹ can also together be the bond fraction of a double bond between the atoms to which they are bonded,
R^{I}, R^{II}, R^{III}, R^{IV} and R^{V}, independently of one another, are hydrogen, alkyl, cycloalkyl, heterocycloalkyl, aryl, hetaryl, hydroxy, thiol, polyalkylene oxide, polyalkyleneimine, alkoxy, halogen, COOH, carboxylate, SO₃H, sulfonate, NE⁷E⁸, alkylene-NE⁷E⁸, trifluoromethyl, nitro, alkoxycarbonyl, acyl or cyano, wherein E⁷ and E⁸ are in each case identical or different radicals selected from hydrogen, alkyl, cycloalkyl and aryl, wherein two adjacent radicals R^{I} to R^{V}, together with the carbon atoms of the benzene ring to which they are bonded, can also be a condensed ring system having 1, 2 or 3 further rings,
Bⁿ⁺ is a mono- to trivalent cation,
n is an integer from 1 to 3,
R² has the meanings given in claim 1,
Z¹ and Z², independently of one another, are hydrogen, alkyl, cycloalkyl, heterocycloalkyl, aryl, heteroaryl, amino, aminocarbonyl, aminothiocarbonyl or thiocarbamidyl, wherein at least one of the radicals Z¹ or Z² is aryl or heteroaryl.

19. The agent according to claim 9, of compounds of the formulae 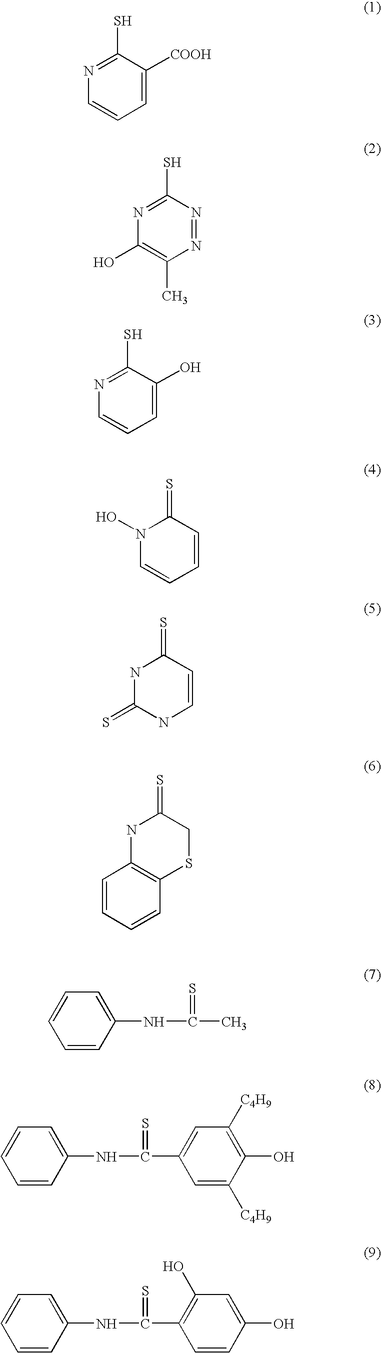 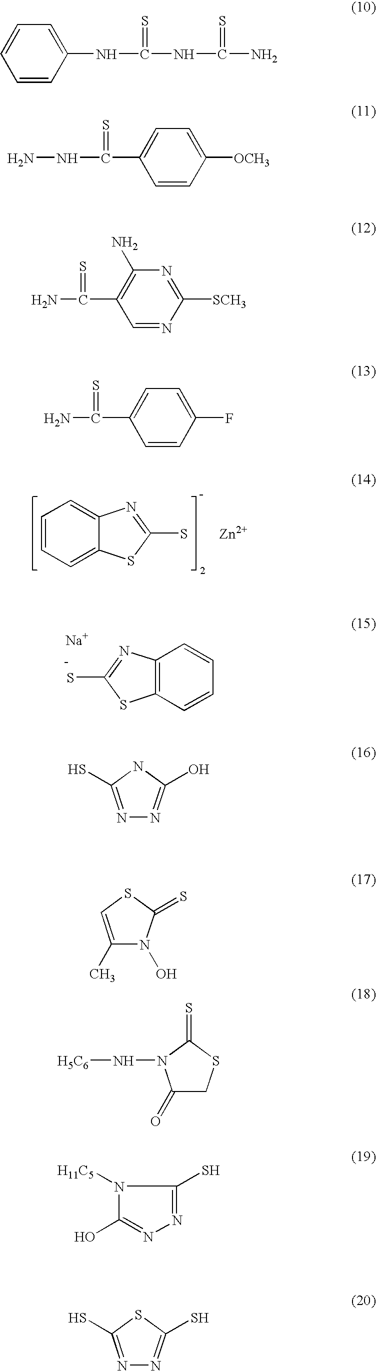 [chemical structure]
